(19) United States
(12) Patent Application Publication
(10) Pub. No.: US 2024/0342159 A1
(43) Pub. Date: Oct. 17, 2024

(54) VALBENAZINE FOR USE IN THE ADD-ON TREATMENT OF SCHIZOPHRENIA

(71) Applicant: Neurocrine Biosciences, Inc., San Diego, CA (US)

(72) Inventor: Angel S. Angelov, San Diego, CA (US)

(21) Appl. No.: 18/575,022

(22) PCT Filed: Jun. 28, 2022
(86) PCT No.: PCT/US2022/073209

§ 371 Date: Dec. 28, 2023

Related U.S. Application Data
(60) Provisional application No. 63/362,561, filed on Apr. 6, 2022.
(60) Provisional application No. 63/276,079, filed on Nov. 5, 2021.
(60) Provisional application No. 63/242,794, filed on Sep. 10, 2021.
(60) Provisional application No. 63/216,875, filed on Jun. 30, 2021.

(51) Int. Cl. A61K 31/4745; A61K 9/48; A61K 31/40; A61K 31/407; A61K 31/451; A61K 31/454; A61K 31/4545; A61K 31/496; A61K 31/519; A61K 31/5415; A61K 31/551; A61K 31/553; A61K 31/554

(52) U.S. Cl. CPC A61K 31/4745; A61K 9/4825; A61K 9/4858; A61K 9/4866; A61K 31/40; A61K 31/407; A61K 31/451; A61K 31/454; A61K 31/4545; A61K 31/496; A61K 31/519; A61K 31/5415; A61K 31/551; A61K 31/553; A61K 31/554

(57) ABSTRACT

Provided herein are methods for the add-on treatment of schizophrenia using a vesicular monoamine transporter isoform 2 (VMAT2) inhibitor. In certain embodiments, the VMAT2 inhibitor is valbenazine or a pharmaceutically acceptable salt thereof.

### BACKGROUND

Schizophrenia is a chronic and severe mental disorder that affects how a person thinks, feels, and behaves. The onset of the disease is usually in late adolescence or early adulthood. Schizophrenia presents with a heterogeneous combination of symptoms typically categorized into positive, negative, and cognitive symptoms.

While no definitive single etiology has been identified, schizophrenia has multifactorial etiological underpinnings including strong genetic contributions and prominent gene environment interactions. Schizophrenia manifests with subtle structural and functional abnormalities of various brain structures, such as the striatum, hippocampus, and prefrontal cortex. Although schizophrenia is highly heritable, as indicated by epidemiological studies, it is not a purely genetic disorder nor is it caused by a single gene. Rather, it likely results from a combination of both rare and common genetic variants, environmental risk factors, and their interactions.

Overall, patients with schizophrenia have higher mortality and morbidity rates, with a reduced expected lifespan that is nearly 20 years shorter than that of the general population.

Pharmacotherapy is the mainstay of schizophrenia management and antipsychotics are generally employed for the treatment of schizophrenia and associated symptoms; however, some patients fail to respond to antipsychotics. Additionally, antipsychotics can have a wide range of side effects and poor compliance with drug treatment is a frequent problem among schizophrenia patients. Non-compliance with antipsychotic medication can lead to relapse for patients in remission and persistent symptoms for those with existing symptoms.

### BRIEF SUMMARY

In an aspect, provided herein is a method for the add-on treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, wherein the subject is also being administered at least one co-therapeutic agent for the treatment of schizophrenia.

In an aspect, provided herein is a method for the add-on treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, wherein the subject has at least one symptom chosen from the following symptoms in Positive and Negative Symptom Scale (PANSS): P1 (delusions), P3 (hallucinations), P6 (suspiciousness), and G9 (unusual thought content), and further wherein the subject is also being administered at least one co-therapeutic agent for the treatment of schizophrenia.

In certain embodiments, the subject has at least one sign or symptom of schizophrenia.

In certain embodiments, the subject has at least one positive, negative, and/or cognitive sign or symptom of schizophrenia.

In certain embodiments, the at least one sign or symptom is delusions, hallucinations, disorganized speech, disorganized behavior or attention, anhedonia, catatonic behavior, affective flattening, alogia, avolition, conceptual disorganization, or any combination thereof.

In certain embodiments, the subject has a total PANSS score ≥70.

In certain embodiments, the subject has Clinical Global Impression of Severity (CGI-S) score ≥4.

In certain embodiments, the subject has a stable background antipsychotic medication dose.

In certain embodiments, the subject has a stable PANSS total score.

In certain embodiments, the subject is residually symptomatic or has at least one residual sign or symptom after first- or second-line treatment of schizophrenia.

In certain embodiments, the subject is residually symptomatic or has at least one residual sign or symptom after mono- or combination therapy for schizophrenia.

In certain embodiments, the mono- or combination therapy is initial therapy.

In certain embodiments, the at least one co-therapeutic agent is least one antipsychotic agent.

In certain embodiments, the antipsychotic agent is a typical antipsychotic agent.

In certain embodiments, the typical antipsychotic agent is benperidol, chlorpromazine, droperidol, flupentixol, fluphenazine, haloperidol, levomepromazine, loxapine, molindone, pericyazine, perphenazine, pimozide, prochlorperazine, promazine, sulpiride, thiothixene, trifluoperazine, thioridazine, zuclopenthixol, or any combination thereof.

In certain embodiments, the antipsychotic agent is an atypical antipsychotic agent.

In certain embodiments, the atypical antipsychotic agent is clozapine, olanzapine, risperidone, sertindole, quetiapine, paliperidone, asenapine, ziprasidone, surmontil, iloperidone, aripiprazole, or any combination thereof.

In certain embodiments, the subject is receiving concomitant treatment with a subtherapeutic dose of a second antipsychotic agent.

In certain embodiments, the concomitant treatment with a subtherapeutic dose of a second antipsychotic is used to treat insomnia or anxiety.

In certain embodiments, the concomitant treatment with a subtherapeutic dose of a second antipsychotic is used to treat symptoms other than refractory positive psychosis symptoms.

In certain embodiments, the subject has an inadequate response to the at least one antipsychotic agent.

In certain embodiments, the subject is on at least a second clinical use of the at least one antipsychotic agent.

In certain embodiments, the subject is also being administered risperidone and/or a risperidone equivalent.

In certain embodiments, the subject is also being administered a total daily dose of from about 4 mg to about 8 mg of risperidone and/or risperidone equivalents.

In certain embodiments, the subject is receiving background antipsychotic therapy at a total daily dose of from about 4 mg to about 8 mg of risperidone equivalents according to Table 2.

In certain embodiments, the subject has a stable antipsychotic agent dose.

In certain embodiments, the stable antipsychotic agent dose is defined as ≤25% change in risperidone equivalent total daily dose.

In certain embodiments, the subject is also being administered up to two antipsychotic agents.

In certain embodiments, the total combined antipsychotic dose is from about 4 mg to about 8 mg daily dose of risperidone equivalents according to Table 2.

In certain embodiments, the antipsychotic agent is other than clozapine.

In certain embodiments, the subject is treated with a stable regimen of antipsychotic agent(s) with no clinically meaningful change in the prescribed dose.

In certain embodiments, the no clinically meaningful change in the prescribed dose is no increase in dose or ≤25% decrease in dose for tolerability.

In certain embodiments, no dose adjustment is anticipated.

In certain embodiments, the subject is also being administered a long-acting injectable antipsychotic.

In certain embodiments, there is no dose change in the long-acting injectable antipsychotic.

In certain embodiments, the inadequate response comprises at least one baseline criterion selected from:
Positive and Negative Symptom Scale (PANSS) total score of ≥70;
PANSS score of ≥4 on at least one symptom selected from P1 (delusions), P3 (hallucinations), P6 (suspiciousness), and G9 (unusual thought content); Clinical Global Impression of Severity (CGI-S) score of ≥4;
stable background antipsychotic medication dose of ≤25% change in risperidone equivalent total daily dose according to Table 2; and
   a stable PANSS total score of ≤15% change.

In certain embodiments, the subject has a confirmed diagnosis of schizophrenia as defined by the MINI Version 7.0.2 for Psychotic Disorders for the Psychotic Disorders for the Diagnostic and Statistical Manual of Mental Disorders, Fifth Edition (DSM-V) for a subject ≥18 years of age or K-SADS-PL for a subject 13 to 17 years of age.

In certain embodiments, the subject has a confirmed initial diagnosis of schizophrenia for at least one year.

In certain embodiments, the subject is at least 13 years of age with a body weight of at least 50 kg.

In certain embodiments, the subject is an outpatient with stable symptomatology

In certain embodiments, the subject does not have comorbid Parkinsonism and/or does not or exhibit more than a minimal level of extrapyramidal signs or symptoms as defined by a score on the modified Simpson Angus Scale (SAS; excluding #10, akathisia) >6.

In certain embodiments, the subject does not have treatment-resistant schizophrenia, as defined by at least one of the following criteria:
a history of clozapine treatment for treatment-resistant psychosis; and/or
a history of multiple adequate and failed antipsychotic medication trials, wherein the subject demonstrated minimal or no improvement.

In certain embodiments, the subject does not have a diagnosis of schizoaffective disorder; a lifetime diagnosis of bipolar disorder; a lifetime diagnosis of obsessive-compulsive disorder; a recent occurrence of panic disorder, a depressive episode, and/or other comorbid psychiatric condition(s) requiring clinical attention based on the MINI Version 7.0.2.

In certain embodiments, the subject does not have evidence of depression as measured by a Calgary Depression Scale for Schizophrenia (CDSS) score >8.

In certain embodiments, the subject has not attempted suicide and/or does not have positive answers on item numbers 4 or 5 on the Columbia-Suicide Severity Rating Scale (C-SSRS).

In certain embodiments, the (S)-2-amino-3-methyl-butyric acid (2R,3R,11 bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered via a titration scheme that comprises the up-titration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester over a period of no more than about six weeks until an optimized dose is administered.

In certain embodiments, the titration scheme comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof at an initial dose equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily for about two weeks, provided that the subject tolerates the initial dose and that the subject has not had an adequate response, increasing the dose and administering the increased dose to the subject.

In certain embodiments, the increased dose is equivalent to about 60 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, the titration scheme further comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof at the increased dose for about two weeks.

In certain embodiments, if the subject does not tolerate the increased dose, the optimized dose is the initial dose.

In certain embodiments, if the subject tolerates the increased dose and if the subject has had an adequate response, the optimized dose is the increased dose.

In certain embodiments, the methods of the present disclosure further comprise administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof to the subject.

In certain embodiments, if the subject tolerates the increased dose and if the subject has not had an adequate response, the method further comprises increasing the dose.

In certain embodiments, the further increased dose is equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, if the subject does not tolerate the further increased dose, the optimized dose is the increased dose.

In certain embodiments, if the subject tolerates the further increased dose and if the subject has had an adequate response, the optimized dose is the further increased dose.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered in the afternoon or evening.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject is treated with a stable regimen of antipsychotic agent(s) with 25% change in risperidone equivalent total daily dose.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject is treated with a stable regimen of antipsychotic agent(s) with no clinically meaningful change in the prescribed dose.

In certain embodiments, the no clinically meaningful change in the prescribed dose is no increase in dose or a ≤25% decrease in dose for tolerability in the prescribed dose for ≥3 weeks.

In certain embodiments, no dose adjustment is anticipated.

In certain embodiments, the subject is also being administered a long-acting injectable antipsychotic with no dose change within 8 weeks prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a Diagnostic and Statistical Manual of Mental Disorders, Fifth Edition (DSM-V) diagnosis of schizophrenia.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a confirmed diagnosis of schizophrenia as defined by the MINI Version 7.0.2 for Psychotic Disorders for the Diagnostic and Statistical Manual of Mental Disorders, Fifth Edition (DSM-V) for a subject ≥18 years of age or K-SADS-PL for a subject 13 to 17 years of age.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a confirmed initial diagnosis of schizophrenia for at least one year.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has at least one symptom chosen from the following symptoms in Positive and Negative Symptom Scale (PANSS): P1 (delusions), P3 (hallucinations), P6 (suspiciousness), and G9 (unusual thought content).

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a total PANSS score ≥70.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a stable PANSS total score with ≤15% change.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a Clinical Global Impression of Severity (CGI-S) score ≥4.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a stable background antipsychotic medication dose with ≤25% change in risperidone equivalent total daily dose according to Table 2.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject is an outpatient with stable symptomatology for ≥3 weeks.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject does not have treatment-resistant schizophrenia, as defined by at least one of the following criteria:
a history of clozapine treatment for treatment-resistant psychosis; and/or
a history of multiple adequate and failed antipsychotic medication trials, wherein the subject demonstrated minimal or no improvement.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject does not have a diagnosis of schizoaffective disorder; a lifetime diagnosis of bipolar disorder; a lifetime diagnosis of obsessive-compulsive disorder; a recent occurrence of panic disorder, a depressive episode, and/or other comorbid psychiatric condition requiring clinical attention based on the MINI Version 7.0.2.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject does not have evidence of depression as measured by a Calgary Depression Scale for Schizophrenia (CDSS) score >8.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has not attempted suicide within one year and/or does not have positive answers on item numbers 4 or 5 on the Columbia-Suicide Severity Rating Scale (C-SSRS).

In certain embodiments, the add-on treatment results in a change in total PANSS score from Analysis Baseline to Week 10; a change in CGI-S of illness from Analysis Baseline to Week 10, a change in Personal and Social Performance (PSP) score from Analysis Baseline to Week 10, a change in VAS scores for the EuroQol 5 Dimensions 5 Levels (EQ-5D-5L; subjects ≥18 years old) or EQ-5D Youth (EQ-5D-Y; subjects 13 to 17 years old) from baseline to Week 10; and/or a change in Sheehan Disability Score (SDS) from baseline to Week 10.

In certain embodiments, the treatment results in a reduction in the subject's CGI-S positive symptoms, relative to the subject's score at baseline.

In certain embodiments, the treatment results in a change in EuroQol 5 Dimensions 5 Levels (EQ-5D-5L) scores from Analysis Baseline to Week 10 and/or a change in Sheehan Disability Score (SDS) from Analysis Baseline to Week 10.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in a solid dosage form.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is orally administered.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in the form of a capsule.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered daily.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily or twice daily.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily.

In certain embodiments, the therapeutically effective amount is an amount equivalent to from about 10 mg to about 90 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to from about 20 mg to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 20 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 40 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 60 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 80 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a free base.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a salt. In certain embodiments, the salt is a tosylate salt.

In certain embodiments, the tosylate salt is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt of structural Formula (I):

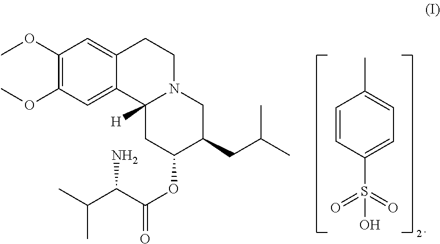

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is in crystalline form.

In certain embodiments, the crystalline form of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is Form I of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt having a differential scanning calorimetric (DSC) peak temperature within 2% of 243° C.

In certain embodiments, the DSC peak temperature is within 1% of 243° C.

In certain embodiments, the DSC peak temperature is within 0.5% of 243° C.

In certain embodiments, the crystalline form has an X-ray powder diffraction (XRPD) pattern comprising a peak at a two-theta angle of 6.3°±0.2°.

In certain embodiments, the crystalline form has an X-ray powder diffraction (XRPD) pattern comprising a peak at a two-theta angle of 17.9°±0.2°.

In certain embodiments, the crystalline form has an X-ray powder diffraction (XRPD) pattern comprising a peak at a two-theta angle of 19.7°±0.2°.

In certain embodiments, the crystalline form is stable upon exposure to about 25° C. and about 60% relative humidity.

In certain embodiments, the crystalline form has a D90 particle size of about 70 μM in length.

In certain embodiments, the crystalline form has a D10 particle size of about 10 μM in length.

In certain embodiments, the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt.

In certain embodiments, the crystalline form has a purity of no less than 98% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt.

In certain embodiments, the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt; and has an X-ray powder diffraction (XRPD) pattern comprising peaks at two-theta angles of 6.3°±0.2°, 17.9°±0.2°, and 19.7°±0.2°.

These and other aspects of the disclosure will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE DRAWING

FIG. 1 shows a schematic of the study design. The study includes a Screening Period of up to 4 weeks, a 2-week double-blind Placebo Run-in Period, an 8 week Randomized Double-Blind Treatment Period, and a 2-week Washout Period. DB=double-blind; V=visit; VBZ=valbenazine; Wk=week.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The present disclosure is directed to methods of treating diseases or disorders relating to dopamine neurotransmission.

Dopamine neurotransmission has been implicated in the underlying pathophysiology of schizophrenia. The dopamine hypothesis of schizophrenia suggests that a dysregulated dopamine system contributes to positive, negative, and cognitive symptoms of the disease. Without wishing to be bound by theory, it is believed that subjects with inadequate response to antipsychotics may be producing dopamine at a higher level, which makes dopamine blockade in the synapse inadequate for controlling symptoms of schizophrenia.

The standard of care in schizophrenia is the use of first-generation (D2 antagonist) and second-generation (D2/HT2 antagonist) antipsychotics for reducing positive symptoms and decreasing the frequency of inpatient hospitalization. However, as many as 20% of subjects with schizophrenia experience treatment failure and are resistant to standard antipsychotics, as defined by lack of clinically important improvement in symptoms after 2 to 3 regimens of treatment with standard antipsychotic drugs. Furthermore, an additional 30% to 40% of patients improve but are residually symptomatic despite antipsychotic treatment. Certain residually symptomatic patients are offered clozapine treatment, however, because of side effects and rigor of follow up for this therapeutic agent, only a relatively small of number of these patients are maintained on the drug. Some patients are tried on two concomitant antipsychotics, yet to a low percentage success rate. Furthermore, currently available antipsychotics fail to return most patients to remission of symptoms.

The present disclosure provides compositions and methods for modulating dopaminergic transmission through inhibition of vesicular monoamine transporter 2 (VMAT2), which mediates monoamine uptake from the cytoplasm to the synaptic vesicle for storage and release, using a VMAT2 inhibitor. Monoamines include, for example, catecholamines (e.g., dopamine, norepinephrine), tryptamines (e.g., serotonin), and histamine. VMAT2 is a transporter which is present in the membrane of presynaptic vesicles in monoaminergic neurons. Their primary function appears to be packaging monoamines (e.g., dopamine) from the cytoplasm of the presynaptic neuron into vesicles for subsequent release into the synapse. Inhibition of VMAT2 results in modulated activity in associated neuronal circuits.

Valbenazine modulates dopaminergic transmission through inhibition of VMAT2, which mediates monoamine uptake from the cytoplasm to the synaptic vesicle for storage and release. In one aspect, unexpectedly a VMAT2 inhibitor, such as valbenazine, acts synergistically with antipsychotics to control schizophrenia symptoms by decreasing dopamine available for release.

In certain embodiments, the adjunctive or add-on treatment of schizophrenia in a subject in need thereof with valbenazine and at least one antipsychotic reduces symptoms, prevents relapse, and/or maximizes quality of life and functioning. In other embodiments, the adjunctive or add-on treatment of schizophrenia in a subject in need thereof with valbenazine and at least one first-generation (D2 antagonist) antipsychotic and/or at least one second-generation (D2/HT2 antagonist) antipsychotic is effective for reducing positive symptoms and/or decreasing the frequency of inpatient hospitalization. In certain embodiments, adjunctive or add-on treatment of individuals with schizophrenia reduces symptoms, prevents relapse, and maximizes quality of life and functioning.

### I. Definitions

In the following description, certain specific details are set forth in order to provide a thorough understanding of various embodiments. However, one skilled in the art will understand that the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, in some embodiments, “pharmaceutically acceptable salt” refers to acid addition salts with an inorganic or an organic acid. Lists of suitable salts are found in WO 87/05297, Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977), each of which is incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002 which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts with an inorganic or an organic base. Inorganic bases which may be used to prepare salts include, for example, sodium, potassium, lithium, ammonium, calcium, magnesium, iron, zinc, manganese, aluminum hydroxides, carbonates, bicarbonates, phosphates, and the like; particularly preferred are the ammonium, potassium, sodium, calcium, and magnesium hydroxides, carbonates, bicarbonates, or phosphates. Organic bases from which may be used to prepare salts include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, “about” means±20% of the stated value, and includes more specifically values of ±10%, ±5%, ±2% and ±1% of the stated value.

As used herein, “co-administer” and “co-administration” and variants thereof mean the administration of at least two drugs (e.g., valbenazine and a co-therapeutic agent) to a patient either sequentially, simultaneously, approximately simultaneously, or consequently proximate in time to one another (e.g., within the same day, or week or period of 30 days, or sufficiently proximate that each of the at least two drugs can be simultaneously detected in the blood plasma). In some embodiments, the two drugs are administered sequentially in any order. In some embodiments, the two drugs are administered simultaneously or approximately simultaneously. In some embodiments, the two drugs are administered sequentially.

When co-administered, two or more active agents can be co-formulated as part of the same composition or administered as separate formulations. This also may be referred to herein as “concomitant” administration or variants thereof.

As used herein, “adjusting administration,” “altering administration,” “adjusting dosing,” or “altering dosing” are all equivalent and mean tapering off, reducing, or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, “baseline” refers to the period of time just prior to initiation of therapy. The patient's condition just prior to initiation of therapy can be referred to as the patient's baseline condition.

As used herein, “deutetrabenazine” may be referred to as (RR, SS)-1, 3, 4, 6, 7, 11b-hexahydro-9, 10-di(methoxyd3)-3-(2-methylpropyl)-2H-benzo[a]quinolizin-2-one. Deutetrabenazine is a racemic mixture containing the following compounds:

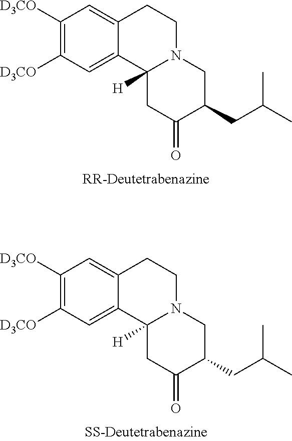

Deutetrabenazine (or d₆-tetrabenazine) as disclosed in U.S. Pat. No. 8,524,733 is administered resulting in an appropriate concentration over a specified period of time of metabolite (+)a-3-isobutyl-9, 10-d6-dimethoxy-1,3,4,6,7,11bhexahydro-2H-pyrido[2, 1-a]isoquinolin-2-ol (deuterated (+)a-HTBZ) or deuterated (+)β-HTBZ in the plasma). The d₆-tetrabenazine may be administered by a variety of methods including the formulations as disclosed in PCT Publication WO 2014/047167, the disclosure of which is incorporated herein by reference in its entirety.

As used herein, dihydrotetrabenazine may be referred to as 2-hydroxy-3-(2-methylpropyl)-1,3,4,6,7,11b-hexahydro-9,10-dimethoxy-benzo(a)quinolizine. The compound has three chiral centers and hence can, theoretically, exist in a total of eight isomeric forms as shown below:

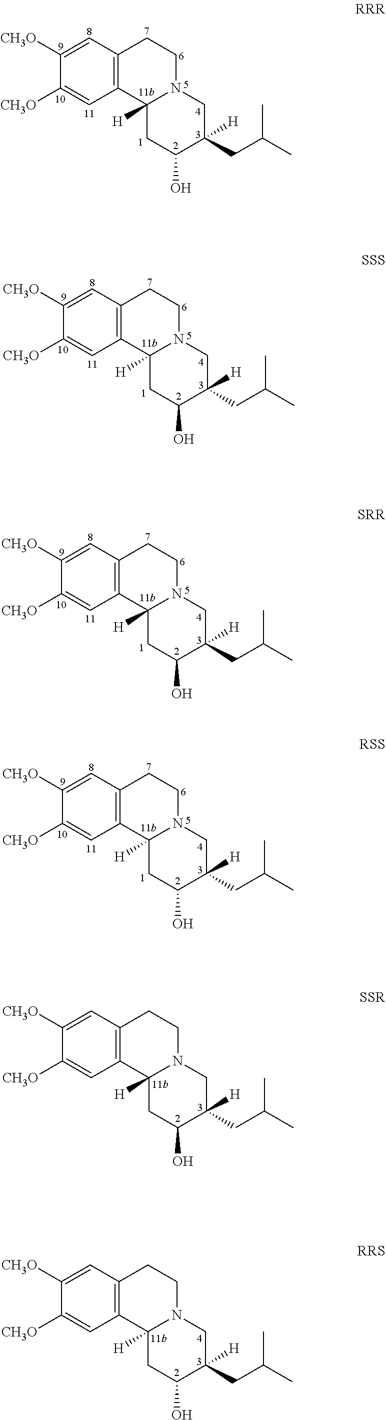

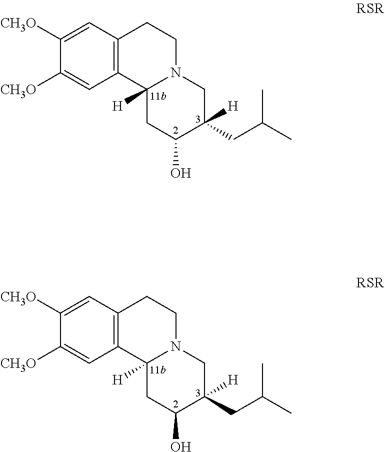

The synthesis and characterization of the eight isomers is described by Sun et al. (2011) Eur. J. Med. Chem. 1841-1848.

As used herein, a “dose” means the measured quantity of an active agent to be taken at one time by a patient. In certain embodiments, wherein the active agent is not valbenazine free base, the quantity is the molar equivalent to the corresponding amount of valbenazine free base. For example, often a drug is packaged in a pharmaceutically acceptable salt form, for example valbenazine ditosylate, and the dosage for strength refers to the mass of the molar equivalent of the corresponding free base, valbenazine. As an example, 73 mg of valbenazine tosylate is the molar equivalent of 40 mg of valbenazine free base.

As used herein, “dosing regimen” means the dose of an active agent taken at a first time by a patient and the interval (time or symptomatic) at which any subsequent doses of the active agent are taken by the patient such as from about 20 to about 160 mg once daily, e.g., about 20, about 40, about 60, about 80, about 100, about 120, or about 160 mg once daily. The additional doses of the active agent can be different from the dose taken at the first time.

As used herein, a “dosage” is the prescribed administration of a specific amount, number, and frequency of doses over a specific period of time.

As used herein, “effective amount” and “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a compound contains an unnatural proportion of deuterium.

With regard to the compounds provided herein, when a particular atomic position is designated as having deuterium or “D” or “d,” it is understood that the abundance of deuterium at that position is substantially greater than the natural abundance of deuterium, which is about 0.015%. A position designated as having deuterium typically has a minimum isotopic enrichment factor of, in certain embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (670.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position. The isotopic enrichment of the compounds provided herein can be determined using conventional analytical methods known to one of ordinary skill in the art, including mass spectrometry, nuclear magnetic resonance spectroscopy, and crystallography.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications set forth in 21 CFR 208, and other applicable regulations, which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, the “Mini International Neuropsychiatric Interview” or “MINI” is a brief structured diagnostic interview for the major psychiatric disorders (including schizophrenia) in the revised DSM Third Edition, DSM Fourth Edition, DSM-5, and International Statistical Classification of Diseases and Related Health Problems, Tenth Edition (ICD-10). See, e.g., Sheehan D V, Lecrubier Y, Sheehan K H, et al. The Mini-International Neuropsychiatric Interview (M.I.N.I.): the development and validation of a structured diagnostic psychiatric interview for DSM-IV and ICD-10. J Clin Psychiatry. 1998; 59 Suppl 20:22-33; quiz 4-57. Validation and reliability studies have been done comparing the MINI to other well-known psychiatric diagnostic interviews. The results of these studies show that the MINI has similar reliability and validity properties to these instruments but can be administered in a much shorter period of time and clinicians can use it after a brief training session. See, e.g., Sheehan D V, Lecrubier Y, Sheehan K H, et al. The validity of the Mini International Neuropsychiatric Interview (MINI) according to the SCID-P and its reliability. Eur Psychiatry. 1997; 12:232-41.

The assessment of the inclusion criterion for schizophrenia will be standardized using the MINI Version 7.0.2. The MINI will also be used to evaluate the presence of comorbid psychiatric disorders to assess the appropriateness of the subject for inclusion.

As used herein, the “Kiddie Schedule for Affective Disorder and Schizophrenia for School-Age Children, Present and Lifetime Version” or “K-SADS-PL” is a semi-structured diagnostic interview in children and adolescents that combines dimensional and categorical assessment approaches to diagnose current and past episodes of psychopathology (e.g., schizophrenia and schizoaffective disorders) in children and adolescents aged 6 to 18 years using DSM-5 criteria. See, e.g., Kaufman J, Birmaher B, Brent D, et al. Schedule for Affective Disorders and Schizophrenia for School-Age Children-Present and Lifetime Version (K-SADS-PL): initial reliability and validity data. J Am Acad Child Adolesc Psychiatry. 1997; 36(7):980-88.

The “Sheehan Disability Scale” or “SDS,” as used herein, is a brief, validated measure of functional impairment in several psychiatric disorders to measure the effect of treatment on disability. See, e.g., Leon A C, Olfson M, Portera L, et al. Assessing psychiatric impairment in primary care with the Sheehan Disability Scale. Int J Psych Med. 1997 27(2):93-105. It includes 3 self-rated items designed to measure how work, social life, and family life are impaired by current psychiatric symptoms. Each item includes an 11-point analog scale that uses visual-spatial, numeric, and verbal descriptive anchors to represent the degree of disruption (0 [none at all] to 10 [extremely]). It also assesses the number of days a subject was unable to work/attend school and the number of days a subject was underproductive in the past week. The SDS will be administered as outlined in the schedule of assessments (see Table 5).

As used herein, the “Positive and Negative Symptoms Scale,” “Positive and Negative Syndrome Scale,” or “PANSS” is a reliable, well known, widely used, clinician administered, validated, 30-item scale designed to evaluate the severity of various symptoms of schizophrenia and is commonly employed in clinical studies involving antipsychotics to measure symptom reduction in patients taking antipsychotics. See, e.g., European Medicines Agency [EMA], Committee for Medicinal Products for Human Use. Guidance on the clinical investigation of medicinal products, including depot preparations in the treatment of schizophrenia. 2012; EMA/CHMP/40072/2010 Rev 1; Lehman A F, Lieberman J A, Dixon L B, et al. Treatment recommendations for patients with schizophrenia. In: Practice Guideline for the Treatment of Patients with Schizophrenia. Second edition. American Psychiatric Association; 2004: 3-35 (Part A); and Kay S R, Fiszbein A, Opler L A. The positive and negative syndrome scale (PANSS) for schizophrenia. Schizophr Bull. 1987; 13(2):261-76.

The subscales of the PANSS and the 5-factor model of the PANSS are commonly employed to assess different symptom domains of schizophrenia. The scale is divided into 3 sections with 7 items designed to evaluate positive symptoms (symptoms of the disease which manifest as the presence of traits), 7 items designed to evaluate negative symptoms (symptoms that manifest as the absence of traits), and 16 items that address general psychopathology. Each item is scored on a 7-point severity scale (1=absent; 2=minimal; 3=mild; 4=moderate; 5=moderate severe; 6=severe; 7=extreme). The scale also includes 3 supplementary items that constitute an aggression risk profile; however, these items will not be scored as they are not applicable to the study. The PANSS total score is derived from the summation of each item.

The PANSS “Positive Symptom Factor Score” or “PSFS” is broadly used in clinical studies of schizophrenia and has demonstrated both good test-retest reliability and validity for positive symptom assessment in patients. See, e.g., Lehman A F, Lieberman J A, Dixon L B, et al. Practice guideline for the treatment of patients with schizophrenia, second edition. Am J Psychiatry. 2004 February; 161(2 Suppl):1-56 and Marder S R, Davis J M, and Chouinard G. The effects of risperidone on the five dimensions of schizophrenia derived from factor analysis: combined results of the North American trials. J Clin Psychiatry. 1997; 58:538-46.

As used herein, the “Personal and Social Performance Scale” or “PSP Scale” is a validated instrument to rate severity of illness on 4 main areas (socially useful activities, personal and social relationships, self-care, and disturbing and aggressive behavior). See, e.g., Morosini et al., 2000. The subject's degree of severity in the 4 domains is rated on a 6-point scale from absent (which means no problems on this dimension), mild, manifest, marked, severe, and very severe. Out of the ratings on the four domains, a single measurement from 0 to 100% can be created. The PSP scale will be administered and scored by the investigator or other qualified site personnel.

As used herein, the “Clinical Global Impression of Severity” or “CGI-S” is a 7-point scale to assess patient's severity of illness. See, e.g., Busner J, Targum S D. The clinical global impressions scale: applying a research tool in clinical practice. Psychiatry (Edgmont). 2007; 4(7):28-37. CGI-S will be administered and scored by the investigator or other qualified site personnel.

As used herein, the “Abnormal Involuntary Movement Scale” or “AIMS” rating scale rates a total of 10 items with 9 items rating involuntary movement from 0 (no dyskinesia) to 4 (severe dyskinesia). Items 1 through 7 comprise facial and oral movements (Items 1 to 4), extremity movements (Items 5 to 6), and trunk movements (Item 7). The AIMS dyskinesia total score for Items 1 to 7 ranges from 0 to 28; a higher score reflects increased severity. Items 8, 9 and 10 rate global judgments with Item 10 being rated based only on the subject's report of his/her awareness of abnormal movements from 0 (no awareness) to 4 (aware, severe distress). Items 11 and 12 are yes/no questions concerning problems with teeth and/or dentures. AIMS total score can range from 0 to 40. See, e.g., Guy W. Ed. ECDEU Assessment Manual of Psychopharmacology, revised, 1976. US Department of Health, Education, and Welfare. Pub. No. (ADM), 76-338. Rockville (MD): National Institute of Mental Health—Clinical Global Impression—Improvement. p. 217-22. The AIMS will be administered by the investigator or other qualified personnel.

As used herein, the “Modified Simpson Angus Scale” or “SAS” is a clinician-administered rating scale that is widely used to assess antipsychotic-induced parkinsonism in clinical practice and research settings. See, e.g., Simpson G M, Angus J W. A rating scale for extrapyramidal side effects. Acta Psychiatr Scand Suppl. 1970; 212:11-9. The study described herein uses a modified 10-item version of the SAS (for the screening and Day 1 assessment of eligibility) in which “Leg Pendulousness” and “Head Dropping” items included in the original version have been replaced with “Head Rotation” and “Akathisia,” which has been used frequently in schizophrenia clinical trials. See, e.g., Moore T J, Furberg C D. The harms of antipsychotic drugs: evidence from key studies. Drug Saf. 2017; 40(1):3-14. Each item is rated using a 5-point scale (0-4); the modified SAS scores can range from 0 to 40.

As used herein, the “Calgary Depression Scale for Schizophrenia” or “CDSS” is used to assess symptoms of major depressive disorder in patients with schizophrenia, and specifically designed to assess comorbid depressive symptoms. See, e.g., Addington D, Addington J, Maticka-Tyndale E. Assessing depression in schizophrenia: the Calgary Depression Scale. Br J Psychiatry Suppl. 1993; (22):39-44. The CDSS consists of 9 items: depressed mood, hopelessness, self-deprecation, guilty ideas of reference, pathological guilt, depression worse in the morning, early wakening, suicide, and observed depression. Each item is rated using a 0 to 3 point scale (0-3); the CDSS score can range from 0 to 27. The items on the CDSS are all typical depressive symptoms and do not appear to overlap with the negative symptoms of schizophrenia. The CDSS will be conducted by the investigator or other qualified site personnel.

As used herein, the “Placebo-Control Reminder Script” or “PCRS” is a script which carefully reviews placebo response factors with research participants with the goal of reducing placebo effect through increased awareness of its impact. The PCRS script is read to each participant prior to the administration of efficacy assessments. It is followed by a discussion between the rater and participant about the key points, including understanding of double-blind clinical trials, recognition of the rater's neutral position regarding participant responses, and the importance of providing honest feedback. The PCRS mitigates expectation bias leading to increased placebo response. See, e.g., Cohen E A, Hassman H H, Ereshefsky L, et al. Placebo response mitigation with a participant focused psychoeducational procedure: a randomized, single-blind, all placebo study in major depressive and psychotic disorders. Neuropsychopharmacology. 2021; 46(4):844-50.

As used herein, the “Columbia-Suicide Severity Rating Scale” or “C-SSRS” is a validated, evidence-supported scale used for suicide assessment and prospectively assesses suicidal ideation and behavior. See, e.g., The World Wide Web at cssrs.columbia.edu. The C-SSRS will be administered and scored by the investigator or qualified study site personnel.

As used herein, the “EuroQol 5 Dimensions 5 Levels” or “EQ-5D-5L” is a general, single index measure for describing and valuing health (Herdman et al., 2011). It defines health in terms of 5 dimensions: Mobility, Self-Care, Usual Activities, Pain/Discomfort, and Anxiety/Depression. Each dimension has 5 levels: no problems, slight problems, moderate problems, severe problems, and extreme problems. The subject indicates his/her health state by checking the box next to the most appropriate statement. The scores for the 5 dimensions can be combined into a 5-digit number that describes the patient's health state. Subjects also rate their overall health on a 0 to 100 hash-marked, vertical visual analogue scale (VAS). The endpoints are labeled “The best health you can imagine” and “The worst health you can imagine.”

The EQ-5D youth (EQ-5D-Y) version is a more comprehensible instrument suitable for children and adolescents. The EQ-5D-Y comprises 2 pages: the EQ-5D descriptive system and the EQ visual analogue scale (EQ VAS). The EQ-5D-Y descriptive system comprises the following five dimensions: mobility, looking after myself, doing usual activities, having pain or discomfort and feeling worried, sad or unhappy. Each dimension has 3 levels: no problems, some problems and a lot of problems. The younger patient is asked to indicate his/her health state by ticking the box next to the most appropriate statement in each of the five dimensions. The EQ VAS records the subjects self-rated health on a vertical visual analogue scale where the endpoints are labeled “The best health you can imagine” and “The worst health you can imagine.” The EQ-5D-5L (subjects ≥18 years old) and the EQ-5D-Y (subjects 13 to 17 years old) will be administered by the investigator or qualified designee.

As used herein, “positive symptoms” and “positive symptoms of schizophrenia” are interchangeable and include, but are not limited to, behaviors and thoughts that are not normally present, such as psychosis, delusions, hallucinations, and disorganized speech and behavior.

As used herein, “negative symptoms” and “negative symptoms of schizophrenia” are interchangeable and include, but are not limited to, motivational syndrome comprising social withdrawal, affective flattening, anhedonia, and diminished energy. See, e.g, Fervaha G, Foussias G, Agid O, et al. Impact of primary negative symptoms on functional outcomes in schizophrenia. Eur Psychiatry. 2014 September; 29(7):449-55.

As used herein, “cognitive symptoms” and “cognitive symptoms of schizophrenia” are interchangeable and include, but are not limited to, poor information processing, impaired ability to focus on objectives, and abnormalities of working memory and learning (Kahn R S, Sommer I E, Murray R M, et al. Schizophrenia. Nat Rev Dis Primers. 2015 November; 1:15067; and van Os J and Kapur S. Schizophrenia. The Lancet. 2009 August; 374:635-45.

As used herein, “The Healthcare Utilization Questionnaire” is a questionnaire designed to evaluate whether a subject had any non-study-related health care provider interactions. The questionnaire includes 3 questions which ask subject about any hospitalizations, emergency room/urgent care visits, and outpatient visits over the past 30 days (at first use) or since the last study visit (at all subsequent uses).

As used herein, “patient” or “individual” or “subject” means a mammal, including a human, for whom or which therapy is desired, and generally refers to the recipient of the therapy.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug Administration. “Pharmacologically active” (or “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, tosylic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

The phrase “an isotopic variant thereof; or a pharmaceutically acceptable salt thereof” as used herein has the same meaning as the phrase “an isotopic variant; or a pharmaceutically acceptable salt of the compound referenced therein; or an isotopic variant; or a pharmaceutically acceptable salt of an enantiomer or a mixture of enantiomers of the compound referenced therein.”

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the terms “VMAT2 inhibitor,” “inhibit VMAT2,” or “inhibition of VMAT2” refer to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The terms and/or phrases “VMAT2 inhibitor,” “inhibit VMAT2,” or “inhibition of VMAT2” also refer to altering the function of VMAT2 by decreasing the probability that a complex forms between a VMAT2 and a natural substrate.

As used herein, “tetrabenazine” may be referred to as 1,3,4,6,7,11b-hexahydro-9,10-dimethoxy-3-(2-methylpropyl)-2H-benzo[a]quinolizin-2-one. The compound has chiral centers at the 3- and 11b-carbon atoms and hence can, theoretically, exist in a total of four isomeric forms as shown below:

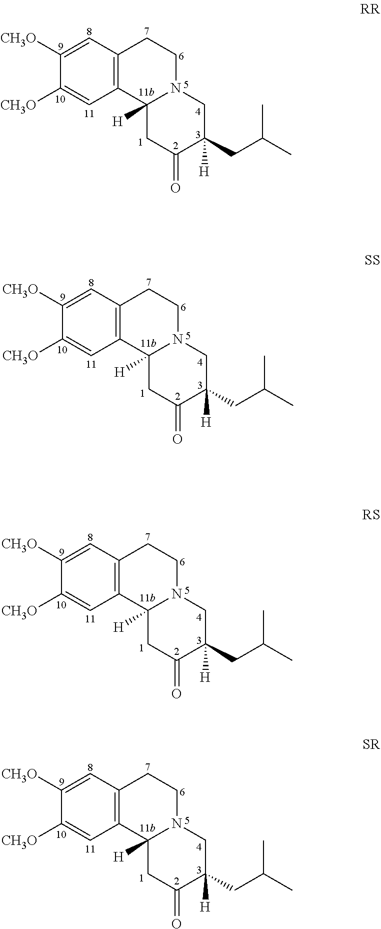

Commercially available tetrabenazine is a racemic mixture of the RR and SS isomers. Tetrabenazine may be administered by a variety of methods including the formulations disclosed in PCT Publications WO 2010/018408, WO 2011/019956, and WO 2014/047167, the disclosure of each of which is incorporated herein by reference in its entirety.

As used herein, “up-titration” of a compound refers to increasing the amount of a compound to achieve a therapeutic effect that occurs before dose-limiting intolerability for the patient. Up-titration can be achieved in one or more dose increments, which may be the same or different.

As used herein, “valbenazine” may be referred to as (S)-2-amino-3-methyl-butyric acid (2R,3R,11bR)-3-isobutyl-9,10-dimethoxy-1,3,4,6,7,11b-hexahydr a]isoquinolin-2-yl ester; or as L-valine, (2R,3R,11bR)-1,3,4,6,7,11b-hexahydro-9,10-dimethoxy-3-(2-methylpropyl)-2H-benzo[a]quinolizin-2-yl ester or as NBI-98854 and has the following chemical structure:

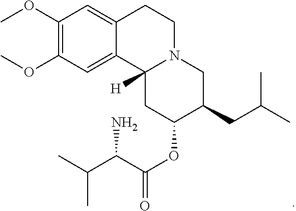 [SMILES: COc1cc2c(cc1OC)[C@H]1C[C@@H](OC(=O)[C@@H](N)C(C)C)[C@H](CC(C)C)CN1CC2]

A formulation of valbenazine:4-toluenesulfonate (1:2) (referred to herein as “valbenazine ditosylate”) has been previously reported in the FDA approved drug label under the trade name INGREZZA®.

Valbenazine can be prepared according to U.S. Pat. Nos. 8,039,627 and 8,357,697, the disclosure of each of which is incorporated herein by reference in its entirety. In certain embodiments, the valbenazine for use in the compositions and methods provided herein is in polymorphic Form I as disclosed in U.S. Pat. No. 10,065,952, the disclosure of which is incorporated herein by reference in its entirety.

### Valbenazine Ditosylate

In another embodiment, provided herein is a crystalline form of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) or an isotopic variant thereof or solvate thereof of Formula I:

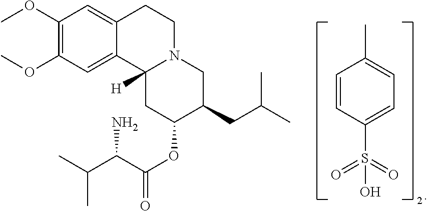

### Valbenazine Ditosylate Form I

In yet another embodiment, provided herein is a crystalline form of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) or an isotopic variant thereof or solvate thereof, wherein the crystalline form is Form I.

In various embodiments, crystalline Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) has an X-ray diffraction pattern. In some embodiments, the X-ray diffraction pattern of Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3, 17.9, and 19.7°. In some embodiments, the X-ray powder diffraction pattern of Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3, 17.9, or 19.7°. In another embodiment, crystalline Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3° and 19.7°. In another embodiment, crystalline Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3°.

In some embodiments, crystalline Form I has one or more characteristic XRP diffraction peaks at two-theta angles of approximately 6.3° and approximately 19.7°. In certain embodiments, crystalline Form I has one or more characteristic XRP diffraction peaks at two-theta angles of approximately 6.3°, approximately 17.9°, and approximately 19.7°. In some embodiments, crystalline Form I has one or more characteristic XRP diffraction peaks at two-theta angles of approximately 6.3°, approximately 17.9°, approximately 19.7°, and approximately 22.7°. In certain embodiments, crystalline Form I has one or more characteristic XRP diffraction peaks at two-theta angles of approximately 6.3°, approximately 15.6°, approximately 17.9°, approximately 19.7°, and approximately 22.7°. In some embodiments, crystalline Form I has one or more characteristic XRP diffraction peaks at two-theta angles of approximately 6.3°, approximately 15.6°, approximately 16.6°, approximately 17.9°, approximately 19.7°, and approximately 22.7°.

In various embodiments, crystalline Form I has an endothermic differential scanning calorimetric (DSC) thermogram. In some embodiments, crystalline Form I has a DSC thermogram comprising an endothermic event with an onset temperature of about 240° C. and a peak at about 243° C. In yet another embodiment, crystalline Form I has a thermal gravimetric analysis (TGA) plot comprising a mass loss of less than about 0.4% when heated from about 25° C. to about 140° C.

In various embodiments, crystalline Form I has a gravimetric vapor system (GVS) plot. In some embodiments, crystalline Form I exhibit a mass increase of about 1% when subjected to an increase in relative humidity from about 0% to about 95% relative humidity. In certain embodiments mass gained upon adsorption is lost when the relative humidity (RH) is decreased back to about 0% RH. In still another embodiment, crystalline Form I is stable upon exposure to about 25° C. and about 60% relative humidity. In yet another embodiment, crystalline Form I is stable upon exposure to about 25° C. and about 60% relative humidity for about 24 months. Also in another embodiment, crystalline Form I is stable upon exposure to about 25° C. and about 60% relative humidity for about 3 months. In still another embodiment, crystalline Form I is stable upon exposure to about 25° C. and about 92% relative humidity. In another embodiment, crystalline Form I is stable upon exposure to about 40° C. and about 75% relative humidity. In another embodiment, crystalline Form I is stable upon exposure to about 40° C. and about 75% relative humidity for about 6 months. In another embodiment, crystalline Form I is stable upon exposure to about 40° C. and about 75% relative humidity for about 3 months.

In certain embodiments, crystalline form of Formula I in Form I may contain no less than about 95%, no less than about 97%, no less than about 98%, no less than about 99%, or no less than about 99.5% by weight of the salt of Formula I. The crystalline form may also contain no less than about 90%, no less than about 95%, no less than about 98%, no less than about 99%, or no less than 99.5% by weight of crystal Form I.

In certain embodiments, crystalline Form I has an aqueous solubility of about 17.58, about 18.58, about 19.58, about 26.75, about 26.87, about 26.96, about 27.06, about 27.75, about 27.87, about 27.97, about 28.06, about 28.75, about 28.87, about 28.97, about 29.06, about 27.45, about 28.45, about 29.45, about 30.61, about 31.61, about 32.61, about 32.17, about 32.98, about 33.17, about 33.98, about 34.17, about 34.35, about 34.98, about 35.35, about 36.35 mg/mL. In certain embodiments, crystalline Form I has an aqueous solubility of about 31.61 and about 33.17 at approximately pH 1.2; about 28.45 and about 27.97 at approximately pH 3; about 28.06 and about 27.77 at approximately pH 4; about 18.58 and about 27.87 at approximately pH 5; about 33.98 and about 35.35 at approximately pH 6.8.

In certain embodiments, crystalline Form I may contain no greater than about 0.1%, no greater than about 0.11%, no greater than about 0.12%, no greater than about 0.13%, no greater than about 0.14%, no greater than about 0.15%, no greater than about 0.16%, no greater than about 0.17%, no greater than about 0.18%, no greater than about 0.19%, no greater than about 0.2%, no greater than about 0.21%, no greater than about 0.22%, no greater than about 0.23%, no greater than about 0.24%, no greater than about 0.25%, no greater than about 0.26%, no greater than about 0.27%, no greater than about 0.28%, no greater than about 0.29%, no greater than about 0.3%, no greater than about 0.31%, no greater than about 0.32%, no greater than about 0.33%, no greater than about 0.34%, no greater than about 0.35%, no greater than about 0.36%, no greater than about 0.37%, no greater than about 0.38%, no greater than about 0.39%, no greater than about 0.4%, no greater than about 0.5%, no greater than about 0.6%, no greater than about 0.7%, no greater than about 0.8%, no greater than about 0.9%, no greater than about 1%, no greater than about 2%, no greater than about 3%, no greater than about 4%, or no greater than about 5% water by weight.

In certain embodiments Form I may be characterized by particle analysis. In certain embodiments, a sample of Form I comprises particles having rhomboid crystal morphology. In yet another embodiment, a sample of Form I comprises particles of about 100, about 90, about 80, about 70, about 60, about 50, about 40, about 30, about 20, about 10, about 5 μM in length. In some embodiments, a sample of Form I comprises particles of about 70, about 60, about 40, about 20, about 10 μM in length. In other embodiments, a sample of Form I comprises particles of about 69.39, about 56.22, about 34.72, about 17.84, about 10.29 μM in length.

### II. Pharmaceutical Compositions

Also provided is a composition for treating a patient in need of a vesicular monoamine transport 2 (VMAT2) inhibitor chosen from valbenazine and (+)-α-3-isobutyl-9,10-dimethoxy-1,3,4,6,7,11b-hexahydro-2H-pyrido[2,1-a]isoquinolin-2-ol, or a pharmaceutically acceptable salt and/or isotopic variant thereof, comprising a therapeutically effective amount of the VMAT2 inhibitor.

In certain embodiments, the composition is for treating a neurological or psychiatric disease or disorder. In certain embodiments, the neurological or psychiatric disease or disorder is schizophrenia. In certain embodiments, the treatment is add-on or adjunctive treatment.

In certain embodiments, the composition is administered in an amount equivalent to from about 20 mg to about 120 mg of valbenazine free base of the VMAT2 inhibitor. In certain embodiments, the composition is administered in an amount equivalent to about 20 mg of valbenazine free base of the VMAT2 inhibitor. In certain embodiments, the composition is administered in an amount equivalent to about 40 mg of valbenazine free base of the VMAT2 inhibitor. In certain embodiments, the composition is administered in an amount equivalent to about 80 mg of valbenazine free base of the VMAT2 inhibitor. In certain embodiments, the composition is administered in an amount equivalent to about 60 mg of valbenazine free base of the VMAT2 inhibitor. In certain embodiments, the composition is administered in an amount equivalent to about 120 mg of valbenazine free base of the VMAT2 inhibitor.

In certain embodiments, the composition is administered for a first period of time in a first amount of the VMAT2 inhibitor and then the amount is increased to a second amount. In certain embodiments, the first period of time is a week. In certain embodiments, the first amount is equivalent to about 40 mg of valbenazine free base. In certain embodiments, the second amount is equivalent to about 80 mg of valbenazine free base.

Also provided herein is a pharmaceutical composition for use in treating neurological or psychiatric disease or disorders, comprising the VMAT2 inhibitor as an active pharmaceutical ingredient, in combination with one or more pharmaceutically acceptable carriers or excipients.

The choice of excipient, to a large extent, depends on factors, such as the particular mode of administration, the effect of the excipient on the solubility and stability of the active ingredient, and the nature of the dosage form.

The pharmaceutical compositions provided herein may be provided in unit dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered alone, or in combination with one or more other compounds provided herein, one or more other active ingredients. The pharmaceutical compositions provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art). The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### III. Methods of Use

Valbenazine can be administered for the add-on treatment of schizophrenia, according to the methods disclosed in U.S. Pat. Nos. 10,857,137; 10,874,648; 10,912,771; 10,940,141; 10,952,997; 10,857,148; and 10,993,941, the disclosure of each of which is incorporated herein by reference in its entirety. Valbenazine can be administered for the add-on treatment of schizophrenia, according to the methods disclosed in U.S. Ser. No. 17/080,343 and U.S. Pat. No. 11,04,029, the disclosure of each of which is incorporated herein by reference in its entirety. In some embodiments, valbenazine is administered for the adjunctive treatment of schizophrenia.

In an aspect, provided herein is a method for the add-on treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, wherein the subject is also being administered at least one co-therapeutic agent for the treatment of schizophrenia.

In an aspect, provided herein is a method for the add-on treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, wherein the subject has at least one symptom chosen from the following symptoms in Positive and Negative Symptom Scale (PANSS): P1 (delusions), P3 (hallucinations), P6 (suspiciousness), and G9 (unusual thought content), and further wherein the subject is also being administered at least one co-therapeutic agent for the treatment of schizophrenia.

In an aspect, provided herein is a method for the adjunctive treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, wherein the subject is also being administered at least one co-therapeutic agent for the treatment of schizophrenia.

In an aspect, provided herein is a method for the adjunctive treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, wherein the subject has at least one symptom chosen from the following symptoms in Positive and Negative Symptom Scale (PANSS): P1 (delusions), P3 (hallucinations), P6 (suspiciousness), and G9 (unusual thought content), and further wherein the subject is also being administered at least one co-therapeutic agent for the treatment of schizophrenia.

In certain embodiments, the subject has at least one sign or symptom of schizophrenia. In certain embodiments, the subject has a return or worsening of symptoms following a period of remission. In certain embodiments, the subject exhibits symptoms of a schizophrenia relapse.

In certain embodiments, the subject has at least one positive, negative, and/or cognitive sign or symptom of schizophrenia.

In certain embodiments, the at least one sign or symptom is delusions, hallucinations, disorganized speech, disorganized behavior or attention, anhedonia, catatonic behavior, affective flattening, alogia, avolition, conceptual disorganization, or any combination thereof.

In certain embodiments, the subject has a total PANSS score ≥70.

In certain embodiments, the subject has Clinical Global Impression of Severity (CGI-S) score ≥4.

In certain embodiments, the subject has a stable background antipsychotic medication dose.

In certain embodiments, the subject has a stable PANSS total score.

In certain embodiments, the subject is residually symptomatic or has at least one residual sign or symptom after first- or second-line treatment of schizophrenia.

In certain embodiments, the subject is residually symptomatic or has at least one residual sign or symptom after mono- or combination therapy for schizophrenia.

In certain embodiments, the mono- or combination therapy is initial therapy.

In certain embodiments, the at least one co-therapeutic agent is least one antipsychotic agent.

In certain embodiments, the antipsychotic agent is a typical antipsychotic agent.

In certain embodiments, the typical antipsychotic agent is benperidol, chlorpromazine, droperidol, flupentixol, fluphenazine, haloperidol, levomepromazine, loxapine, molindone, pericyazine, perphenazine, pimozide, prochlorperazine, promazine, sulpiride, thiothixene, trifluoperazine, thioridazine, zuclopenthixol, or any combination thereof.

In certain embodiments, the antipsychotic agent is an atypical antipsychotic agent.

In certain embodiments, the atypical antipsychotic agent is clozapine, olanzapine, risperidone, sertindole, quetiapine, paliperidone, asenapine, ziprasidone, surmontil, iloperidone, aripiprazole, or any combination thereof.

In certain embodiments, the subject is receiving concomitant treatment with a subtherapeutic dose of a second antipsychotic agent.

In certain embodiments, the concomitant treatment with a subtherapeutic dose of a second antipsychotic is used to treat specific symptoms, such as insomnia or anxiety.

In certain embodiments, the concomitant treatment with a subtherapeutic dose of a second antipsychotic is used to treat symptoms other than refractory positive psychosis symptoms.

In certain embodiments, the subject has an inadequate response to the at least one antipsychotic agent.

In certain embodiments, the subject is on at least a second clinical use of the at least one antipsychotic agent.

In certain embodiments, the subject is also being administered risperidone and/or a risperidone equivalent.

In certain embodiments, the subject is also being administered a total daily dose of from about 4 mg to about 8 mg of risperidone and/or risperidone equivalents.

In certain embodiments, the subject is receiving background antipsychotic therapy at a total daily dose of from about 4 mg to about 8 mg of risperidone equivalents according to Table 2.

In certain embodiments, the subject has a stable antipsychotic agent dose.

In certain embodiments, the stable antipsychotic agent dose is defined as 25% change in risperidone equivalent total daily dose.

In certain embodiments, the subject is also being administered up to two antipsychotic agents.

In certain embodiments, the total combined antipsychotic dose is from about 4 mg to about 8 mg daily dose of risperidone equivalents according to Table 2.

In certain embodiments, the antipsychotic agent is other than clozapine.

In certain embodiments, the subject is treated with a stable regimen of antipsychotic agent(s) with no clinically meaningful change in the prescribed dose.

In certain embodiments, the no clinically meaningful change in the prescribed dose is no increase in dose or ≤25% decrease in dose for tolerability.

In certain embodiments, no dose adjustment is anticipated.

In certain embodiments, the subject is also being administered a long-acting injectable antipsychotic.

In certain embodiments, there is no dose change in the long-acting injectable antipsychotic.

In certain embodiments, the inadequate response comprises at least one baseline criterion selected from:
Positive and Negative Symptom Scale (PANSS) total score of ≥70;
PANSS score of ≥4 on at least one symptom selected from P1 (delusions), P3 (hallucinations), P6 (suspiciousness), and G9 (unusual thought content);
Clinical Global Impression of Severity (CGI-S) score of ≥4;
stable background antipsychotic medication dose of ≤25% change in risperidone equivalent total daily dose according to Table 2; and
a stable PANSS total score of ≤15% change.

In certain embodiments, the subject has a confirmed diagnosis of schizophrenia as defined by the MINI Version 7.0.2 for Psychotic Disorders for the Psychotic Disorders for the Diagnostic and Statistical Manual of Mental Disorders, Fifth Edition (DSM-V) for a subject ≥18 years of age or K-SADS-PL for a subject 13 to 17 years of age.

In certain embodiments, the subject has a confirmed initial diagnosis of schizophrenia for at least one year.

In certain embodiments, the subject is at least 13 years of age with a body weight of at least 50 kg.

In certain embodiments, the subject is under 18 years of age. In certain embodiments, the subject is ≥13 years of age. In certain embodiments, the subject is from 12 to 17 years of age. In certain embodiments, the subject is from 18 to 64 years of age. In certain embodiments, the subject is ≥65 years of age.

In certain embodiments, the subject is an outpatient with stable symptomatology

In certain embodiments, the subject does not have comorbid Parkinsonism and/or does not or exhibit more than a minimal level of extrapyramidal signs or symptoms as defined by a score on the modified Simpson Angus Scale (SAS; excluding #10, akathisia) >6.

In certain embodiments, the subject does not have treatment-resistant schizophrenia, as defined by at least one of the following criteria:
a history of clozapine treatment for treatment-resistant psychosis; and/or
a history of multiple adequate and failed antipsychotic medication trials, wherein the subject demonstrated minimal or no improvement. In some embodiments, failure to tolerate a medication does not constitute failure to respond.

In certain embodiments, the subject does not have a diagnosis of schizoaffective disorder; a lifetime diagnosis of bipolar disorder; a lifetime diagnosis of obsessive-compulsive disorder; a recent occurrence of panic disorder, a depressive episode, and/or other comorbid psychiatric condition(s) requiring clinical attention based on the MINI Version 7.0.2. In certain embodiments, the subject has a historical prior lifetime diagnosis of schizoaffective disorder, but the subject's overall history and current clinical presentation and history is most consistent with schizophrenia, not schizoaffective disorder.

In certain embodiments, the subject does not have evidence of depression as measured by a Calgary Depression Scale for Schizophrenia (CDSS) score >8.

In certain embodiments, the subject has not attempted suicide and/or does not have positive answers on item numbers 4 or 5 on the Columbia-Suicide Severity Rating Scale (C-SSRS).

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered via a titration scheme that comprises the up-titration of the (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester over a period of no more than about six weeks until an optimized dose is administered.

In certain embodiments, the titration scheme comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof at an initial dose equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily for about two weeks, provided that the subject tolerates the initial dose and that the subject has not had an adequate response, increasing the dose and administering the increased dose to the subject.

In certain embodiments, the increased dose is equivalent to about 60 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, the titration scheme further comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof at the increased dose for about two weeks.

In certain embodiments, if the subject does not tolerate the increased dose, the optimized dose is the initial dose.

In certain embodiments, if the subject tolerates the increased dose and if the subject has had an adequate response, the optimized dose is the increased dose.

In certain embodiments, the methods of the present disclosure further comprise administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof to the subject.

In certain embodiments, if the subject tolerates the increased dose and if the subject has not had an adequate response, the method further comprises increasing the dose.

In certain embodiments, the further increased dose is equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, if the subject does not tolerate the further increased dose, the optimized dose is the increased dose.

In certain embodiments, if the subject tolerates the further increased dose and if the subject has had an adequate response, the optimized dose is the further increased dose.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered in the afternoon or evening.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject is treated with a stable regimen of antipsychotic agent(s) with 25% change in risperidone equivalent total daily dose.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject is treated with a stable regimen of antipsychotic agent(s) with no clinically meaningful change in the prescribed dose.

In certain embodiments, the no clinically meaningful change in the prescribed dose is no increase in dose or a ≤25% decrease in dose for tolerability in the prescribed dose for ≥3 weeks.

In certain embodiments, no dose adjustment is anticipated.

In certain embodiments, the subject is also being administered a long-acting injectable antipsychotic with no dose change within 8 weeks prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a Diagnostic and Statistical Manual of Mental Disorders, Fifth Edition (DSM-V) diagnosis of schizophrenia.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a confirmed diagnosis of schizophrenia as defined by the MINI Version 7.0.2 for Psychotic Disorders for the Diagnostic and Statistical Manual of Mental Disorders, Fifth Edition (DSM-V) for a subject ≥18 years of age or K-SADS-PL for a subject 13 to 17 years of age.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a confirmed initial diagnosis of schizophrenia for at least one year.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has at least one symptom chosen from the following symptoms in Positive and Negative Symptom Scale (PANSS): P1 (delusions), P3 (hallucinations), P6 (suspiciousness), and G9 (unusual thought content).

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a total PANSS score ≥70.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a stable PANSS total score with 15% change.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a Clinical Global Impression of Severity (CGI-S) score ≥4.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has a stable background antipsychotic medication dose with ≤25% change in risperidone equivalent total daily dose according to Table 2.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject is an outpatient with stable symptomatology for ≥3 weeks.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject does not have treatment-resistant schizophrenia, as defined by at least one of the following criteria:
a history of clozapine treatment for treatment-resistant psychosis; and/or
a history of multiple adequate and failed antipsychotic medication trials, wherein the subject demonstrated minimal or no improvement. In some embodiments, failure to tolerate a medication does not constitute failure to respond.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject does not have a diagnosis of schizoaffective disorder; a lifetime diagnosis of bipolar disorder; a lifetime diagnosis of obsessive-compulsive disorder; a recent occurrence of panic disorder, a depressive episode, and/or other comorbid psychiatric condition requiring clinical attention based on the MINI Version 7.0.2.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject does not have evidence of depression as measured by a Calgary Depression Scale for Schizophrenia (CDSS) score >8.

In certain embodiments, prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, the subject has not attempted suicide within one year and/or does not have positive answers on item numbers 4 or 5 on the Columbia-Suicide Severity Rating Scale (C-SSRS).

In certain embodiments, the add-on treatment results in a change in total PANSS score from Analysis Baseline to Week 10; a change in CGI-S of illness from Analysis Baseline to Week 10, a change in Personal and Social Performance (PSP) score from Analysis Baseline to Week 10, a change in VAS scores for the EuroQol 5 Dimensions 5 Levels (EQ-5D-5L; subjects ≥18 years old) or EQ-5D Youth (EQ-5D-Y; subjects 13 to 17 years old) from baseline to Week 10; and/or a change in Sheehan Disability Score (SDS) from baseline to Week 10.

In certain embodiments, the treatment results in a reduction in the subject's CGI-S positive symptoms, relative to the subject's score at baseline.

In certain embodiments, the treatment results in a change in EuroQol 5 Dimensions 5 Levels (EQ-5D-5L) scores from Analysis Baseline to Week 10 and/or a change in Sheehan Disability Score (SDS) from Analysis Baseline to Week 10.

In some embodiments, valbenazine is administered orally once daily (QD) as adjunctive treatment in subjects with schizophrenia who have had an inadequate response to antipsychotics.

In some embodiments, administration of valbenazine orally once daily (QD) as adjunctive treatment in a subject with schizophrenia who has had an inadequate response to antipsychotics increases dose stability of background antipsychotic medication(s) in the subject.

In some embodiments, administration of valbenazine orally once daily (QD) as adjunctive treatment in a subject with schizophrenia who has had an inadequate response to antipsychotics increases treatment compliance and symptom stability in the subject. In some embodiments, administration of valbenazine orally once daily (QD) as adjunctive treatment in a subject with schizophrenia who has had an inadequate response to antipsychotics results in subject remission or returns the subject to remission. In certain embodiments of the methods for the adjunctive therapy of schizophrenia disclosed herein, the subject has a return or worsening of symptoms following a period of remission. In certain embodiments, the remission is a level of symptomology that does not interfere with a subject's behavior and/or is also below that required for a diagnosis of schizophrenia.

In certain embodiments of the methods for the adjunctive therapy of schizophrenia disclosed herein, the subject exhibits symptoms of a schizophrenia relapse. In certain embodiments of the methods for the adjunctive therapy of schizophrenia disclosed herein, the therapy prevents or avoids a relapse.

In some embodiments, administration of valbenazine orally once daily (QD) as adjunctive treatment in a subject with schizophrenia who has had an inadequate response to antipsychotics increases stability of schizophrenia symptomology, treatment compliance, and/or dose stability of background antipsychotic medication(s) in the subject.

In some embodiments, administration of valbenazine orally once daily (QD) as adjunctive treatment in subjects with schizophrenia who has had an inadequate response to antipsychotics, wherein the valbenazine is administered at an initial dose for a period of time followed by a scheduled dose increase. In certain embodiments, the period of time for the initial dose is a week. In certain embodiments, the initial dose is equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base. In certain embodiments, the increased dose is equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base. In certain embodiments, the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in a solid dosage form.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is orally administered.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in the form of a capsule.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered daily.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily or twice daily.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily.

In certain embodiments, the therapeutically effective amount is an amount equivalent to from about 10 mg to about 90 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to from about 20 mg to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 20 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 40 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 60 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 80 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a free base.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a salt. In certain embodiments, the salt is a tosylate salt.

In certain embodiments, the tosylate salt is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt of structural Formula (I):

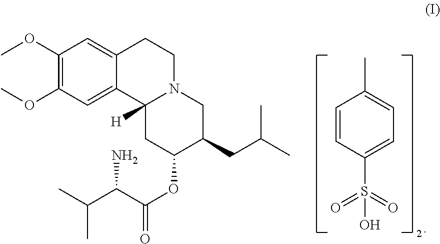

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is in crystalline form.

In certain embodiments, the crystalline form of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is Form I of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt having a differential scanning calorimetric (DSC) peak temperature within 2% of 243° C.

In certain embodiments, the DSC peak temperature is within 1% of 243° C.

In certain embodiments, the DSC peak temperature is within 0.5% of 243° C.

In certain embodiments, the crystalline form has an X-ray powder diffraction (XRPD) pattern comprising a peak at a two-theta angle of 6.3°±0.2°.

In certain embodiments, the crystalline form has an X-ray powder diffraction (XRPD) pattern comprising a peak at a two-theta angle of 17.9°±0.2°.

In certain embodiments, the crystalline form has an X-ray powder diffraction (XRPD) pattern comprising a peak at a two-theta angle of 19.7°±0.2°.

In certain embodiments, the crystalline form is stable upon exposure to about 25° C. and about 60% relative humidity.

In certain embodiments, the crystalline form has a D90 particle size of about 70 μM in length.

In certain embodiments, the crystalline form has a D10 particle size of about 10 μM in length.

In certain embodiments, the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt.

In certain embodiments, the crystalline form has a purity of no less than 98% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt.

In certain embodiments, the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt; and has an X-ray powder diffraction (XRPD) pattern comprising peaks at two-theta angles of 6.3°±0.2°, 17.9°±0.2°, and 19.7°±0.2°.

In some embodiments, provided herein is method for the add-on treatment of schizophrenia, wherein the patient is also being administered a strong cytochrome P450 2D6 (CYP2D6) inhibitor, comprising:
orally administering once daily to the patient a vesicular monoamine transport 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, in an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the add-on treatment of schizophrenia, wherein the patient is a cytochrome P450 2D6 (CYP2D6) poor metabolizer, comprising:
orally administering once daily to the patient a vesicular monoamine transporter 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, in an amount of equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the add-on treatment of schizophrenia, comprising:
(a) orally administering to the patient a therapeutically effective amount of a vesicular monoamine transport 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof,
(b) subsequently determining that the patient is a cytochrome P450 2D6 (CYP2D6) poor metabolizer; and
(c) reducing dosage of the VMAT2 inhibitor administered to the patient to an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the add-on treatment of schizophrenia, comprising:
determining if the patient is a poor metabolizer of cytochrome P450 2D6 (CYP2D6); and
if the patient is a poor metabolizer of cytochrome P450 2D6 (CYP2D6), then orally administering to the patient a first therapeutically effective amount of a vesicular monoamine transport 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, wherein the first therapeutically effective amount is an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester; or
if the patient is not a poor metabolizer of cytochrome P450 2D6 (CYP2D6), then orally administering to the patient a second therapeutically effective amount of a vesicular monoamine transport 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, wherein the second therapeutically effective amount is an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester for one week, and subsequently administering an increased amount of the VMAT2 inhibitor after one week.

In some embodiments, provided herein is a method for the add-on treatment of schizophrenia, comprising:
(a) orally administering to the patient a therapeutically effective amount of a vesicular monoamine transport 2 (VMAT2) inhibitor which is a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, wherein the therapeutically effective amount is an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
(b) subsequently determining that the patient is a poor metabolizer of cytochrome P450 2D6 (CYP2D6); and
(c) administering the same therapeutically effective amount of the VMAT2 inhibitor to the patient.

In some embodiments, provided herein is a method for the add-on treatment of schizophrenia, wherein the patient is a cytochrome P450 2D6 (CYP2D6) poor metabolizer, comprising:
orally administering once daily to the patient a therapeutically effective amount of a vesicular monoamine transporter 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof.

In some embodiments, provided herein is a method of administering a vesicular monoamine transporter (VMAT2) inhibitor chosen from valbenazine and (+)-α-3-isobutyl-9,10-dimethoxy-1,3,4,6,7,11b-hexahydro-2H-pyrido[2,1-a]isoquinolin-2-ol, or a pharmaceutically acceptable salt thereof, or an isotopic variant thereof, to a patient in need thereof, wherein the patient is being treated with a strong cytochrome P450 3A4 (CYP3A4) inducer, comprising:
discontinuing treatment of the strong CYP3A4 inducer and then administering the VMAT2 inhibitor to the patient, thereby avoiding the use of the VMAT2 inhibitor in combination with the strong CYP3A4 inducer.

In some embodiments, provided herein is a method for the add-on treatment of schizophrenia in a patient in need thereof, wherein the patient is being administered a strong cytochrome P450 3A4 (CYP3A4) inducer, comprising:
discontinuing treatment of the strong CYP3A4 inducer, and then
orally administering once daily to the patient a therapeutically effective amount of a vesicular monoamine transporter 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and a pharmaceutically acceptable salt thereof, thereby avoiding the concomitant use of the VMAT2 inhibitor with the strong CYP3A4 inducer.

In some embodiments, provided herein is a method for the add-on treatment of schizophrenia, wherein the patient is also being administered a strong cytochrome P450 3A4 (CYP3A4) inhibitor, comprising:
orally administering once daily to the patient a vesicular monoamine transporter 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, in an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for the add-on treatment of schizophrenia in a patient, comprising:
(a) orally administering to the patient a therapeutically effective amount of a vesicular monoamine transporter 2 (VMAT2) inhibitor chosen from (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof,
(b) subsequently determining that the patient is being administered a strong cytochrome P450 3A4 (CYP3A4) inhibitor; and
(c) reducing dosage of the VMAT2 inhibitor administered to the patient to an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester once daily.

In some embodiments, provided herein is a method for the add-on treatment of schizophrenia in a patient, comprising:
(a) orally administering to the patient a therapeutically effective amount of a vesicular monoamine transporter 2 (VMAT2) inhibitor which is a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, wherein the therapeutically effective amount is an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester once daily;
(b) subsequently determining that the patient is being administered a strong cytochrome P450 3A4 (CYP3A4) inhibitor; and
(c) administering the same therapeutically effective amount of the VMAT2 inhibitor to the patient.

In some embodiments, provided herein is a method for the add-on treatment of schizophrenia, wherein the patient is also being co-administered digoxin, comprising:
(a) administering to the patient a therapeutically effective amount of a vesicular monoamine transport 2 (VMAT2) inhibitor chosen from (S)-2-amino-3-methyl-butyric acid (2R,3R,11bR)-3 sobutyl-9,10-dimethoxy-1,3,4,6,7,11b-hexahydro-2H-pyrido[2,1-a]isoquinolin-2-yl ester, (+)-α-3-isobutyl-9,10-dimethoxy-1,3,4,6,7,11b-hexahydro-2H-pyrido[2,1-a]isoquinolin-2-ol, and pharmaceutically acceptable salts and isotopic variants thereof,
(b) monitoring the digoxin concentration in the patient's blood; and
(c) reducing the dose of digoxin when the digoxin exposure in the patient's blood is increased as compared with the digoxin level in a patient who is administered digoxin alone.

In some embodiments, provided herein is a method for the add-on treatment of schizophrenia, wherein the patient is also in need of treatment with digoxin, the method comprising:
orally administering to the patient a therapeutically effective amount of a vesicular monoamine transport 2 (VMAT2) inhibitor which is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester ditosylate, and
administering the digoxin to the patient at a reduced dose to compensate for the expected increase in exposure resulting from co-administration of the digoxin and the VMAT2 inhibitor,
wherein the reduced dose is relative to what the patient would be administered if the patient is not being administered the VMAT2 inhibitor.

In some embodiments, a method for the adjunctive treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, wherein the subject is also being administered at least one co-therapeutic agent for the treatment of schizophrenia is provided herein.

Provided herein is a method for the adjunctive treatment of schizophrenia in a patient in need thereof, comprising administering an effective amount of a vesicular monoamine transport 2 (VMAT2) inhibitor.

Also provided is a method for the adjunctive treatment of one or more symptoms of schizophrenia in a patient in need thereof, comprising administering an effective amount of a vesicular monoamine transport 2 (VMAT2) inhibitor.

Also provided is a method for the treatment of residual symptoms in a patient in need thereof, comprising administering to the patient a therapeutically effective amount of vesicular monoamine transport 2 (VMAT2) inhibitor.

In some embodiments, the VMAT2 inhibitor is chosen from valbenazine, or a pharmaceutically acceptable salt and/or isotopic variant thereof. In some embodiments, the VMAT2 inhibitor is valbenazine, or a pharmaceutically acceptable salt thereof. In some embodiments, the VMAT2 inhibitor is a valbenazine tosylate salt. In some embodiments, the VMAT2 inhibitor is a ditosylate salt of valbenazine.

In some embodiments, the VMAT2 inhibitor is an isotopic variant that is L-valine, (2R,3R,11bR)-1,3,4,6,7,11b-hexahydro-9,10-di(methoxy-d3)-3-(2-methylpropyl)-2H-benzo[a]quinolizin-2-yl ester or a pharmaceutically acceptable salt thereof.

In some embodiments, the VMAT2 inhibitor is tetrabenazine (9,10-dimethoxy-3-isobutyl-1,3,4,6,7,11b-hexahydro-2H-pyrido[2,1-a]isoquinolin-2-one), or a pharmaceutically acceptable salt and/or isotopic variant thereof. In some embodiments, tetrabenazine is chosen from the RR, SS, RS, and SR isomers of tetrabenazine, and mixtures thereof. In some embodiments, tetrabenazine is a mixture of the RR and SS isomers.

In some embodiments, the VMAT2 inhibitor is deutetrabenazine.

In some embodiments, the VMAT2 inhibitor is chosen from dihydrotetrabenazine (2-hydroxy-3-(2-methylpropyl)-1,3,4,6,7,11b-hexahydro-9,10-dimethoxy-benzo(a)quinolizine), or a pharmaceutically acceptable salt and/or isotopic variant thereof. In some embodiments, dihydrotetrabenazine is chosen from the RRR, SSS, SSRR, RSS, SSR, RRS, RSR, and SRS isomers of dihydrotetrabenazine, and mixtures thereof. In some embodiments, the VMAT2 inhibitor is the RRR isomer ((+)-α-3-isobutyl-9,10-dimethoxy-1,3,4,6,7,11b-hexahydro-2H-pyrido[2,1-a]isoquinolin-2-ol), or a pharmaceutically acceptable salt and/or isotopic variant thereof.

In some embodiments, the patient is also being administered one or more antipsychotic medications for the treatment of schizophrenia. In some embodiments, the patient has an inadequate response to the one or more antipsychotic medications. In some embodiments, the patient is on at least a second clinical use of one or more antipsychotic medications.

In some embodiments, the VMAT2 inhibitor is administered via a titration scheme that comprises the up-titration of the VMAT2 inhibitor over a period of no more than about six weeks until an optimized dose is administered.

In some embodiments, the titration scheme comprises administering the VMAT2 inhibitor at an initial dose equivalent to about 40 mg of valbenazine free base once daily for about two weeks, provided that the patient tolerates the initial dose and that the patient has not had an adequate response, increasing the dose, and administering the increased dose to the patient.

In some embodiments, the increased dose is equivalent to about 60 mg of valbenazine free base once daily.

In some embodiments, the titration scheme further comprises administering the VMAT2 inhibitor at said increased dose for about two weeks.

In some embodiments, if the patient does not tolerate the increased dose, the optimized dose is the initial dose.

In some embodiments, if the patient tolerates the increased dose and if the patient has had an adequate response, the optimized dose is the increased dose.

In some embodiments, the method further comprises administering the optimized dose of the VMAT2 inhibitor to the patient.

In some embodiments, if the patient tolerates the increased dose and if the patient has not had an adequate response, the method further comprises increasing the dose.

In some embodiments, the further increased dose is equivalent to about 80 mg of valbenazine free base once daily.

In some embodiments, if the patient does not tolerate the further increased dose, the optimized dose is the increased dose.

In some embodiments, if the patient tolerates the further increased dose and if the patient has had an adequate response, the optimized dose is the further increased dose.

In some embodiments, the method further comprises administering the optimized dose of the VMAT2 inhibitor to the patient.

In some embodiments, safety of the treatment is measured by one or more of the following assessments: the Extrapyramidal Symptom Rating Scale—(ESRS-A) and the Columbia-Suicide Severity Rating Scale (C-SSRS). In some embodiments, safety of the treatment is measured by one or more of PANSS, CGI-S, PSP, EQ-5D-5L (or EQ-5D-Y), SAS, and SDS.

In some embodiments, the Brief Adherence Rating scale (BARS scale) is used to monitor study treatment and/or background antipsychotic adherence. In some embodiments, a medication adherence mobile application is used to monitor study treatment and/or background antipsychotic adherence.

In one embodiment, provided herein is a method for the treatment inhibition of human vesicular monoamine transporter isoform 2 (VMAT2), comprising administering to a subject a therapeutically effective amount of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) in an amorphous form, or crystalline Form I, II, III, IV, V, or VI; or an isotopic variant thereof, or solvate thereof.

In one embodiment, provided herein is a method for the treatment inhibition of human vesicular monoamine transporter isoform 2 (VMAT2), comprising administering to a subject a therapeutically effective amount of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (Formula II) in an amorphous form, or crystalline Form I, or II; or an isotopic variant thereof; or solvate thereof.

### Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pregelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Vee gum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL®200 (W. R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant. Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation. The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric coated tablets are compressed tablets coated with substances that resist the action of stomach acid but dissolve or disintegrate in the intestine, thus protecting the active ingredients from the acidic environment of the stomach. Enteric coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems.

The pharmaceutical compositions provided herein may be provided as noneffervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide. Coloring and flavoring agents can be used in all of the above dosage forms. The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H2-receptor antagonists.

### Dosages

In the treatment of schizophrenia or other conditions, disorders or diseases associated with VMAT2 inhibition, an appropriate dosage level generally is about 0.001 to 100 mg per kg patient body weight per day (mg/kg per day), about 0.01 to about 80 mg/kg per day, about 0.1 to about 50 mg/kg per day, about 0.5 to about 25 mg/kg per day, or about 1 to about 20 mg/kg per day, which may be administered in single or multiple doses. Within this range the dosage may be 0.005 to 0.05, 0.05 to 0.5, or 0.5 to 5.0, 1 to 15, 1 to 20, or 1 to 50 mg/kg per day. In certain embodiments, the dosage level is about 0.001 to 100 mg/kg per day.

In certain embodiments, the dosage level is about from 20 to 100 mg/kg per day. In certain embodiments, the dosage level is about 0.01 to about 40 mg/kg per day. In certain embodiments, the dosage level is about 0.1 to about 80 mg/kg per day. In certain embodiments, the dosage level is about 0.1 to about 50 mg/kg per day. In certain embodiments, the dosage level is about 0.1 to about 40 mg/kg per day. In certain embodiments, the dosage level is about 0.5 to about 80 mg/kg per day. In certain embodiments, the dosage level is about 0.5 to about 40 mg/kg per day. In certain embodiments, the dosage level is about 0.5 to about 25 mg/kg per day. In certain embodiments, the dosage level is about 1 to about 80 mg/kg per day. In certain embodiments, the dosage level is about 1 to about 75 mg/kg per day. In certain embodiments, the dosage level is about 1 to about 50 mg/kg per day. In certain embodiments, the dosage level is about 1 to about 40 mg/kg per day. In certain embodiments, the dosage level is about 1 to about 25 mg/kg per day. In certain embodiments, the dosage level is about 1 to about 20 mg/kg per day.

In certain embodiments, the dosage level is about from 5.0 to 150 mg per day, and in certain embodiments from 10 to 100 mg per day. In certain embodiments, the dosage level is about 80 mg per day. In certain embodiments, the dosage level is about 40 mg per day.

For oral administration, the pharmaceutical compositions can be provided in the form of tablet or capsule containing 1.0 to 1,000 mg of the active ingredient, particularly about 1, about 5, about 10, about 15, about 20, about 25, about 30, about 40, about 45, about 50, about 75, about 80, about 100, about 150, about 200, about 250, about 300, about 400, about 500, about 600, about 750, about 800, about 900, and about 1,000 mg of the active ingredient for the symptomatic adjustment of the dosage to the patient to be treated. In certain embodiments, the pharmaceutical compositions can be provided in the form of tablet or capsule containing about 100 mg of the active ingredient. In certain embodiments, the pharmaceutical compositions can be provided in the form of tablet or capsule containing about 80 mg of the active ingredient. In certain embodiments, the pharmaceutical compositions can be provided in the form of tablet or capsule containing about 60 mg of the active ingredient. In certain embodiments, the pharmaceutical compositions can be provided in the form of tablet or capsule containing about 50 mg of the active ingredient. In certain embodiments, the pharmaceutical compositions can be provided in the form of tablet or capsule containing about 40 mg of the active ingredient. In certain embodiments, the pharmaceutical compositions can be provided in the form of tablet or capsule containing about 20 mg of the active ingredient. In certain embodiments, the pharmaceutical compositions can be provided in the form of tablet or capsule containing about 25 mg of the active ingredient. In certain embodiments, the pharmaceutical compositions can be provided in the form of tablet or capsule containing about 20 mg of the active ingredient. The compositions may be administered on a regimen of 1 to 4 times per day, including once, twice, three times, and four times per day. In certain embodiments, the pharmaceutical compositions are administered on a regimen of once per day.

It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds provided herein may also be combined or used in combination with other agents useful in the treatment, prevention, or amelioration of one or more symptoms of the diseases or conditions for which the compounds provided herein are useful, and other conditions commonly treated with antipsychotic medication.

In certain embodiments, the compounds provided herein may also be combined or used in combination with a typical antipsychotic drug. In certain embodiments, the typical antipsychotic drug is fluphenazine, haloperidol, loxapine, molindone, perphenazine, pimozide, sulpiride, thioridazine, or trifluoperazine. In certain embodiments, the antipsychotic drug is an atypical antipsychotic drug. In certain embodiments, the atypical antipsychotic drug is clozapine, aripiprazole, asenapine, iloperidone, olanzapine, paliperidone, quetiapine, risperidone, or ziprasidone.

Such other agents, or drugs, may be administered, by a route and in an amount commonly used thereof, simultaneously, or sequentially with the compounds provided herein. When compounds provided herein are used contemporaneously with one or more other drugs, a pharmaceutical composition containing such other drugs in addition to the compounds provided herein may be utilized but is not required. Accordingly, the pharmaceutical compositions provided herein include those that also contain one or more other active ingredients or therapeutic agents, in addition to the compounds provided herein.

The weight ratio of the compounds provided herein to the second active ingredient may be varied and will depend upon the effective dose of each ingredient. Generally, an effective dose of each will be used. Thus, for example, when the compounds provided herein are used in combination with the second drug, or a pharmaceutical composition containing such other drug, the weight ratio of the particulates to the second drug may range from about 1,000:1 to about 1:1,000, or about 200:1 to about 1:200.

Combinations of the particulates provided herein and other active ingredients will generally also be within the aforementioned range, but in each case, an effective dose of each active ingredient should be used.

In certain embodiments, the VMAT2 inhibitor is administered orally.

In certain embodiments, the VMAT2 inhibitor is administered in the form of a capsule.

In certain embodiments, the VMAT2 inhibitor is administered with or without food.

The compounds provided herein may also be combined or used in combination with other agents useful in the treatment, prevention, or amelioration of one or more symptoms of the diseases or conditions for which the compounds provided herein are useful.

In one embodiment, the compounds provided herein may also be combined or used in combination with other agents useful in the treatment of schizophrenia.

Such other agents, or drugs, may be administered, by a route and in an amount commonly used thereof, simultaneously or sequentially with the compounds provided herein. When the particulates provided herein are used contemporaneously with one or more other drugs, a pharmaceutical composition containing such other drugs in addition to the compounds provided herein may be utilized but is not required. Accordingly, the pharmaceutical compositions provided herein include those that also contain one or more other active ingredients or therapeutic agents, in addition to the compounds provided herein.

The weight ratio of the compounds provided herein to the second active ingredient may be varied and will depend upon the effective dose of each ingredient. Generally, an effective dose of each will be used. Thus, for example, when the compounds provided herein are used in combination with the second drug, or a pharmaceutical composition containing such other drug, the weight ratio of the particulates to the second drug may range from about 1,000:1 to about 1:1,000, or about 200:1 to about 1:200. Combinations of the particulates provided herein and other active ingredients will generally also be within the aforementioned range, but in each case, an effective dose of each active ingredient should be used.

Exemplary embodiments of the present disclosure are provided in the following examples. The following examples are presented only by way of illustration and to assist one of ordinary skill in using the disclosure. The examples are not intended in any way to otherwise limit the scope of the disclosure.

### IV. EXAMPLES

### Example 1: A Phase 3, Randomized, Double-Blind, Placebo-Controlled Study to Evaluate the Efficacy, Safety, and Tolerability of Valbenazine as Adjunctive Treatment in Subjects with Schizophrenia

The efficacy, safety, and tolerability of valbenazine for the adjunctive treatment of schizophrenia in human subjects will be evaluated according to a Phase 3, randomized, double-blind, placebo-controlled clinical study.

Study Objectives: The primary objective for this study is to evaluate the effect of adjunctive valbenazine versus placebo on symptoms of schizophrenia in subjects who have inadequate response to antipsychotic treatment. The secondary objectives for this study are to (1) evaluate the effect of adjunctive valbenazine versus placebo on illness severity and subject functioning in subjects who have inadequate response to antipsychotic treatment; and (2) evaluate the safety and tolerability of valbenazine as adjunctive treatment in subjects who have inadequate response to antipsychotic treatment. The study design is as follows.

Valbenazine will be administered orally once daily (QD) as adjunctive treatment in subjects with schizophrenia who have had an inadequate response to antipsychotic treatment. The subjects will subsequently be evaluated.

Schizophrenia will be defined by the Diagnostic and Statistical Manual of Mental Disorders fifth major revision (DSM-5) and confirmed by the Mini International Neuropsychiatric Interview (MINI [Version 7.0.2]; for subjects ≥18 years of age) or the Kiddie Schedule for Affective Disorders and Schizophrenia, Present and Lifetime version (K-SADS-PL; for subjects 13 to 17 years of age). The MINI will be administered by a healthcare professional with a clinically relevant qualification (e.g., psychiatrist, psychiatric nurse, or psychologist) and documented experience assessing patients with schizophrenia. Raters must be trained and certified for administration of MINI in this study. The MINI will be administered by the investigator or other qualified site personnel at Screening to confirm diagnosis of schizophrenia in subjects 18 years of age or older. The K-SADS-PL will be administered by the investigator or other qualified site personnel at Screening to confirm diagnosis of schizophrenia in subjects 13 to 17 years of age (inclusive).

Duration of Study Treatment and Study Participation: The study includes a Screening Period of up to 4 weeks, a 2-week blinded Placebo Run-in Period, an 8-week Randomized Double-Blind Treatment Period, and a 2-week Washout Period. The expected duration of study participation for each subject is approximately 16 weeks, including the Screening Period of up to 4 weeks, 2-week blinded Placebo Run-in Period, 8-week Randomized Double-Blind Treatment Period, and 2-week Washout Period.

All subjects will receive placebo for 2 weeks during the blinded Placebo Run-in Period. Randomized subjects will receive study treatment (valbenazine or placebo) QD starting at Week 3 until the end of the Randomized Double-Blind Treatment Period. Subjects will receive study treatment for a total of 10 weeks (2 weeks of placebo and 8 weeks of randomized study treatment).

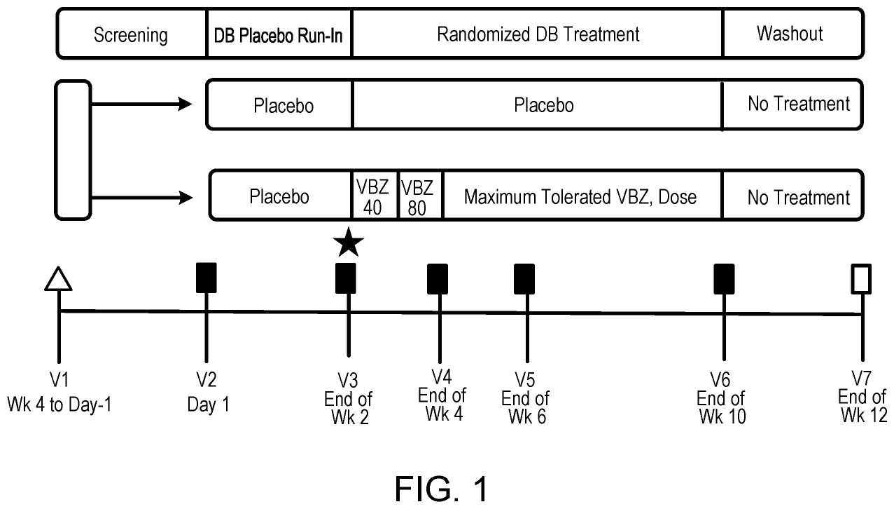

Eligible subjects will receive placebo orally once daily (QD) during a blinded Placebo Run-In Period (Day 1 to end of Week 2), which will be used to evaluate compliance with study treatment and stability of schizophrenic symptoms. At the end of the Placebo Run-In Period (Visit 3), all subjects will be randomized 1:1 to receive valbenazine or placebo orally QD during the 8 week Randomized Double-Blind Treatment Period (Week 3 to the end of Week 10). A two-week Washout Period will be conducted after the final dose of study treatment (end of Week 12). A schematic of the study design is shown in FIG. 1. A list of study assessments to be performed at the study site during the study periods and study visits is provided in the Schedule of Assessments (Table 5).

To maintain the site blind for the blinded Placebo Run-In Period, two versions of the study protocol will be prepared:
An unblinded protocol, which provides details about the blinded Placebo Run-In Period; and
A blinded version of the protocol for submission to the clinical study sites, wherein the blinded Placebo Run-In Period is masked to study site staff and subjects and included as part of the Randomized Double-Blind Treatment Period.

### Screening Period (Week-4 to Day-1)

During a Screening Period of up to 4 weeks, all subjects will be assessed for study eligibility. Subjects must provide written informed consent (or assent for minors per local requirements) before any study-related procedures can be performed. The informed consent form (ICF) will be reviewed with subjects or legal representative (for assent for minors). The ICF must be signed prior to the start of any screening procedures.

After signing the ICF, a subject registry database check must occur to confirm that a subject is not enrolling in multiple clinical studies; this check should be performed as early as possible at the screening visit (Visit 1) to avoid unnecessary procedures. Informed consent for this database check is included in the ICF.

Subjects who provide informed consent and meet a current diagnosis of schizophrenia, as defined by the Mini International Neuropsychiatric Interview (MINI; for subjects ≥18 years old) or Kiddie Schedule for Affective Disorders and Schizophrenia, Present and Lifetime version (K-SADS-PL; for subjects 13 to 17 years old), will undergo additional screening assessments to determine eligibility

Subjects will undergo screening procedures that may be completed in 1 or more visits during the Screening Period of up to 4 weeks. Vital signs should be collected prior to the collection of blood for clinical laboratory tests.

Informant participation at the screening visit is preferred. The informant must provide written informed consent and participate in an in-person interview during the Screening Period.

The medication adherence mobile application must be downloaded at the screening visit, as adherence to background antipsychotic therapy will be assessed throughout the screening period. Subjects using a daily oral antipsychotic must be registered in the platform at screening (Visit 1) and are asked to confirm appropriate adherence to their background antipsychotic each day throughout the Screening Period (Visit 1) to Day 1 (Visit 2). Subjects using a long-acting injectable antipsychotic should be registered in the platform at screening (Visit 1) and will use it to record study treatment intake only.

Sites will be asked to obtain available pharmacy records during the screening period. Training should be provided on the use of concomitant medications.

During the Screening Period, the Sponsor or designee will conduct a site-independent review of selected study entry criteria based on data from the screening visit (Visit 1) provided by the site.

Subjects must be taking at least 1 (with a maximum of 2) antipsychotic medication(s), with a total daily dose between 4 mg and 8 mg of risperidone equivalents (see Table 2), and at least 1 of these antipsychotic medications must be on the approved list and detectable in the subjects' plasma (see Table 3).

Only subjects who are in good health, able to complete the study in the opinion of the investigator and meet the criteria for study eligibility will be enrolled in the study.

Subjects who meet the criteria for study eligibility should continue to receive their current antipsychotic medication(s) at a stable dose for the duration of the study. Stable background antipsychotic medication dose will be defined as subjects who have ≤25% change in dose. Subjects who are unable to maintain a stable antipsychotic dose between the Screening Period (Visit 1) and Day 1 (Visit 2) will be considered screen failures and not able to continue into the study. Eligible subjects will return to the study site on Day 1 for study assessments.

### Screen Failures, Rescreening, and Screening Period

Subjects who do not meet antipsychotic medication stability criteria or other entry criteria during the Screening Period will be considered a screen failure. A subject may be considered for rescreening once with the approval of the Sponsor or designee.

The Screening Period may be extended for certain unavoidable circumstances (such as the COVID-19 pandemic) with approval from Sponsor or designee after discussion of individual circumstances.

### Blinded Placebo Run-In (Day 1 to end of Week 2)

After screening, eligible subjects will be enrolled in the study and enter an investigator/site and subject blinded 2-week Placebo Run-in Period on Day 1 (Visit 2). The blinded Placebo Run-in Period will be used to evaluate stability of schizophrenia symptomology, study treatment compliance, and dose stability of background antipsychotic medication(s).

### Day 1 (Visit 2) Cannot not be Conducted Until:

The antipsychotic plasma assay results have been reviewed by the investigator and deemed acceptable;
Informant consent has been obtained; and
Sponsor or designee has approved subject eligibility (including from site-independent review of select criteria).

Eligible subjects will return to the study site on Day 1 (Visit 2) for study assessments (Table 5). Before the subject can begin study treatment on Day 1 (Visit 2), the investigator must ensure that the subject continues to meet select study entry criteria.

Blood samples for PK assessments taken on Day 1 must be drawn pre-dose.

At Day 1 (Visit 2), subject status in the medication adherence mobile application must be updated to also monitor study treatment intake throughout the blinded Placebo Run-In Period (Day 1 to end of Week 2) and Randomized Double-Blind Treatment Period (Week 3 to end of Week 10) and continue to log their background daily oral antipsychotic use through end of Week 10 (Visit 6).

After completion of all Day 1 (Visit 2) assessments, subjects who continue to meet eligibility criteria will receive placebo capsules that appear identical to the study treatment to be used during the Randomized Double-Blind Treatment Period. Subjects will take their first dose on Day 1. Subjects will be instructed to take 2 capsules QD for 14 days (Day 1 to end of Week 2). Subjects will take 2 blinded placebo capsules orally QD from Day 1 to the end of Week 2. Subjects who are unable to tolerate study treatment during the Placebo Run-In will be reduced to 1 placebo capsule daily until the next study visit. This will be considered a dose reduction; only 1 dose reduction is allowable during the study (Day 1 through end of Week 10).

At the end of the Placebo Run-In Period, treatment compliance and symptom stability will determine eligibility for inclusion in the Efficacy Analysis Set.

### Randomized Double-Blind Treatment Period (Week 3 to End of Week 10)

Approximately 400 subjects will be randomized 1:1 to either the valbenazine or placebo treatment arm. Subjects will be randomized via an interactive web response system (IWRS).

Randomization will be stratified by the following factors:
Region (US versus non-US)
Age (<18 years, 18 to 40 years, and >40 years)

Subjects will be randomized 1:1 at the end of Week 2 (Visit 3) to receive valbenazine or placebo QD for 8 weeks. Subjects will continue to take 2 capsules of study treatment orally starting at Week 3 through the end of the Randomized Double-Blind Treatment Period (end of Week 10 [Visit 6]).

Randomized study treatment will then be administered orally QD in a blinded fashion throughout an 8-week Randomized Double-Blind Treatment Period (Week 3 to the end of Week 10). Subjects randomized to the valbenazine treatment arm will receive an initial dose of 40 mg for the first week of randomized study treatment (Week 3), followed by a scheduled dose increase to 80 mg at Week 4 (see Table 1 for maximum study treatment dose per study period).

Subjects who underwent a dose reduction during the Placebo Run-In and who are subsequently randomized to valbenazine on Visit 3 will be taking 40 mg valbenazine through the duration of the study. Subjects will return to the study site at the end of Weeks 4 (Visit 4), 6 (Visit 5), and 10 (Visit 6).

At each visit, subjects will undergo assessments as outlined in Table 5. Key efficacy assessments performed during this period will include PANSS, CGI-S, PSP, EQ-5D-5L (or EQ-5D-Y), and SDS outcome measures. Key safety assessments performed during this period will include evaluation of vital signs, ECGs, clinical laboratory tests, physical examination, concomitant medications, and AEs; as well as safety assessments using C-SSRS and SAS. In addition, blood samples will be obtained for PK evaluation of valbenazine.

TABLE 1
Maximum Study Treatment Dose Per Study Period

| | Placebo | | | |
|---|---|---|---|---|
| | Run-In | Randomized Double-Blind Treatment Period | | |
| | Week | | | |
| | Day 1 | | | Week |
| | to End | | | 5 through |
| | of Week 2 | Week 3 | Week 4 | end Week 10 |
| Placebo | Placebo | Placebo | | |
| Valbenazine | Placebo | VBZ | VBZ | VBZ |
| | | 40 mg | 80 mg | 80 mg |

VBZ = valbenazine

The subject's dose level may be reduced only once during the study (including the Placebo Run-In Period). Subjects who remain intolerant to study treatment after a dose reduction will be discontinued from study treatment and followed through to the end of Week 10. A subject's dose may only be reduced between Day 1 (Visit 2) and the end of Week 6 (Visit 5). No dose changes will be allowed during the last 4 weeks of study treatment.

Study treatment will be self-administered in the afternoon or evening starting on Day 1; study treatment and background antipsychotic adherence will be monitored with a mobile technology system. Study treatment should be administered at approximately the same time each day during the study.

Study visits should occur at approximately the same time of day as the Day 1 visit to standardize the time of day for the assessment of efficacy, safety, and drug exposure. Visits will have a window of ±3 days.

### Washout Period/Follow-Up Period (Week 11 to End of Week 12)

After the last dose of study treatment (end of Week 10), subjects will have a 2-week Washout Period and attend a final follow-up visit at the end of the 2 weeks (end of Week 12; Visit 7). Subjects will return to the study site 14 days after their last dose of study treatment for the safety follow-up visit (end of Week 12 [Visit 7]). Key safety assessments performed at this visit include evaluation of vital signs and AEs; as well as the C-SSRS.

There are versions of the C-SSRS questionnaire designed for use at screening (Baseline/Screening version) and at baseline and visits throughout the study (Since Last Visit version). All versions of the C-SSRS include a series of screening questions related to suicidal ideation and suicidal behavior. Subject responses of “yes” to one or more screening questions will prompt additional questions that evaluate frequency and intensity of suicidal ideation and/or behavior. Subjects with any suicidal behavior or suicidal ideation of type 4 (active suicidal ideation with some intent to act, without specific plan) or type 5 (active suicidal ideation with specific plan and intent) within 1 year before screening (based on the C-SSRS) or on Day 1 are excluded.

### Study Treatment Period (Day 1 to End of Week 10)

Adherence to background daily oral antipsychotic medications will be assessed throughout study treatment (Day 1 [Visit 2] to end of Week 10 [Visit 6]). Subjects using a long-acting injectable antipsychotic will use the medication adherence mobile application to record study treatment intake only. Subjects will receive a daily background medication questionnaire via the mobile application.

Adherence to study treatment (valbenazine or placebo) will be monitored with the medication adherence mobile application throughout the blinded Placebo Run-In (Day 1 through end of Week 2) and Randomized Double-Blind Treatment Period (Week 3 to end of Week 10).

Informant interviews will be required for the PANSS assessments; informants may provide input by attending onsite visits or via virtual visits.

If the subject has taken any restricted concomitant medications (see Table 7) within 4 hours of Visits 2 through 6 then the site should delay or reschedule these study visit assessments within the window defined in the Schedule of Assessments (see Table 5). The PANSS will be administered and scored by the investigator or other qualified site personnel. The subject must have an adult informant who will be able to provide input for completing PANSS who spends ≥2 hours/week with the subject and is considered reliable by the investigator; refer to inclusion criterion #11 for additional details. The total time to administer the PANSS is approximately 45 minutes.

The PANSS assessments will be audio and video recorded throughout the study. Assessments performed at the screening visit, and other visits at the Sponsor's discretion, will be reviewed by a third party to ensure adequacy of ratings. Confirmation of acceptability of PANSS rating and feedback must be reviewed by study personnel prior to Day 1 (Visit 2).

### Risperidone Equivalent Doses

Table 2 summarizes the approximate dose equivalency of currently marketed antipsychotic medications (for all participating countries) that may be taken as treatments during the study. The total dose of antipsychotic medication(s) must be between 4 mg and 8 mg risperidone equivalents. The information in Table 2 has been derived from published literature, prescription guidance documents, and product labeling; it should be considered an approximation of the dose equivalence between the referenced medications. See, e.g., Gardner D M, Murphy A L, O'Donnell H, Centorrino F, Baldessarini R J. International consensus study of antipsychotic dosing. American Journal of Psychiatry. 2010; 167(6):686-93; Leucht S, Samara M, Heres S, Patel M X, Furukawa T, Cipriani A, et al. Dose equivalents for second-generation antipsychotic drugs: the classical mean dose method. Schizophrenia bulletin. 2015; 41(6):1397-402; and Rothe P H, Heres S, Leucht S. Dose equivalents for second generation long-acting injectable antipsychotics: The minimum effective dose method. Schizophrenia research. 2017.

In subjects aged 13 to 17 years, a lower risperidone equivalent dose may be acceptable following consultation with Sponsor or designee.

Doses of background antipsychotic medication should not exceed the dose range listed in product labeling in the country where the subject is enrolled. Subjects must be taking at least one antipsychotic medication listed in Table 3.

TABLE 2
Risperidone Equivalent Doses

| | Marketed Dose | Equivalent Marketed Dose of Antipsychotics: mg/day | | | | | | |
|---|---|---|---|---|---|---|---|---|
| Generic Name | Strengths (mg) | (oral) or mg/listed administration interval (injectable) | | | | | | |
| Risperidone | 0.25, 0.5, 1, 2, 3, 4, 5 | 0.5 | 1 | 1.5 | 2 | 3 | 4 | 6 |
| Amisulpride | 50, 100, 200, 400 | 50 | 100 | 150 | 200 | 300 | 450 | 600 |
| Aripiprazole | 2, 5, 10, 15, 20, 30 | | 5 | | 10 | 15 | 20 | 30 |
| Aripiprazole | 300, 400 vials | | | | 300 | | 400 | |
| extended release | | | | | | | | |
| (monthly injection) | | | | | | | | |
| Aripiprazole lauroxil | 441, 662, 882 prefilled | | | | 441 | 662 | 882 | |
| extended-release | syringes | | | | | | | |
| injection (monthly | | | | | | | | |
| injection) | | | | | | | | |
| Asenapine | 5, 10 | | | 5 | | 10 | | 20 |
| Brexpiprazole | 0.25, 0.5, 1, 2, 3, 4 | | 0.5 | | 1 | | 2 | 4 |
| Cariprazine | 1.5, 3, 4.5, 6 | | | 1.5 | | 3 | 4.5 | 6 |
| Chlorpromazine | 25, 50, 100, 200 | 50 | 100 | 150 | 200 | 300 | 400 | 600 |
| Chlorprothixene | 10, 15, 25, 50, 100 | | | 125 | 150 | 250 | | 500 |
| Clotiapine | 40 mg (tablets) 100 | 20 | 40 | 60 | 80 | 120 | 160 | 240 |
| | mg/mL (oral solution) | | | | | | | |
| Flupenthixol or | 0.5, 1, 3, 5 | 1 | | 3 | 4 | 6 | | 12 |
| Flupentixol | | | | | | | | |
| Flupenthixol or | 20 mg/mL, 100 mg/mL | | | 12 | 16 | 24 | 32 | 50 |
| Flupentixol | | | | | | | | |
| decanoate (every 2 | | | | | | | | |
| weeks injection) | | | | | | | | |
| Fluphenazine | 1, 2.5, 5, 10 | 1 | 2 | | 4 | 6 | 8 | 12 |
| Fluphenazine | 25 mg/mL | | | | | | 25 | 50 |
| decanoate (monthly | | | | | | | | |
| injection) | | | | | | | | |
| Haloperidol | 0.5, 1, 2, 5, 10 | | | 2.5 | 3.5 | 5 | 7.5 | 10 |
| Haloperidol | 50, 100 mg/mL | | | | 50 | | 100 | 150 |
| decanoate (monthly | | | | | | | | |
| injection) | | | | | | | | |
| Iloperidone | 1, 2, 4, 6, 8, 10, 12 | | 4 | | 8 | 12 | 16 | 24 |
| Levomepromazine | 6, 25, 100 | | | 100 | 125 | 200 | | 400 |
| Loxapine | 5, 10, 25, 50 | 5 | 15 | 20 | 30 | 45 | 65 | 95 |
| Lurasidone | 20, 40, 80, 120 | | 20 | | 40 | | 80 | 120 |
| Melperone | 10, 25, 100 | | 75 | 150 | 225 | 300 | 450 | 500 |
| Mesoridazine | 10, 25, 50, 100 | 25 | 50 | 75 | 100 | 150 | 200 | 300 |
| Olanzapine | 2.5, 5, 7.5, 10, 15, 20 | | 2.5 | 5 | 7.5 | 10 | 15 | 20 |
| Olanzapine pamoate | 210, 310, 405 vials | | | | | 210 | 300 | |
| (every 2 weeks | | | | | | | | |
| injection) | | | | | | | | |
| Olanzapine pamoate | 310, 405 vials | | | | | 300 | 405 | |
| (monthly injection) | | | | | | | | |
| Paliperidone | 1.5, 3, 6, 9, 12 | | 1.5 | | 3 | 4.5 | 6 | 9 |
| Paliperidone | 39, 78, 117, 234 | | 39 | | 78 | 117 | 156 | 234 |
| palmitate (monthly | prefilled syringes | | | | | | | |
| injection) | | | | | | | | |
| Paliperidone | 273, 410, 546, 819 | | | | 273 | 410 | 546 | 819 |
| palmitate (every 3 | prefilled syringes | | | | | | | |
| mo. Injection) | | | | | | | | |
| Perphenazine | 2, 4, 8, 16 | 2 | 6 | 8 | 12 | 16 | 24 | 32 |
| Pimozide | 1, 2 | | 1 | 2 | | 4 | 5 | 8 |
| Pipamperone | 2.5, 10, 25, 40 | 40 | 75 | 100 | 150 | 200 | 300 | 400 |
| Quetiapine | 25, 50, 100, 200, 400 | 50 | 100 | 200 | 250 | 375 | 500 | 800 |
| (including XR | | | | | | | | |
| formulation) | | | | | | | | |
| Risperidone LA | 12.5, 25, 37.5, 50 vials | | | 12.5 | | 25 | 37.5 | 50 |
| (every 2 weeks | | | | | | | | |
| injection) | | | | | | | | |
| Sulpiride | 50, 200, 400 | | 100 | 200 | 300 | 400 | 600 | 800 |
| Thiothixene | 1, 2, 5, 10 | 2 | 4 | 6 | 8 | 12 | 16 | 25 |
| Tiapride | 100, 200 | | 100 | 200 | 300 | 400 | 600 | 800 |
| Trifluoperazine | 1, 2, 5, 10 | | 5 | | 10 | 15 | 20 | 30 |
| Ziprasidone | 20, 40, 60, 80 | | 20 | 40 | 60 | | 120 | 160 |
| Zuclopenthixol | 10, 25, 40 | | | | 20 | 25 | | 50 |
| Zuclopenthixol | 200 mg/mL | | | | | 200 | | 400 |
| decanoate (every 2 | | | | | | | | |
| weeks injection) | | | | | | | | |

### Background Antipsychotic Medications

Table 3 summarizes the approved and permitted background antipsychotic medications that can be detected in subject plasma for this study.

TABLE 3
Approved Background Antipsychotic Medications

| Generic Name | Brand Name(s) |
|---|---|
| Risperidone | Risperdal |
| | Rispolept |
| | Diaforin |
| | Arketin |
| Amisulpride | Solian |
| | Midora |
| | Deniban |
| Aripiprazole | Abilify |
| | Apaloz |
| | Arizol |
| | Abilify extended |
| | release |
| Aripiprazole lauroxil extended-release | Aristada |
| injection (monthly injection) | |
| Asenapine | Saphris |
| | Sycrest |
| Brexpiprazole | Rexulti |
| Cariprazine | Vraylar |
| Chlorpromazine^{a} | Thorazine |
| | Largactil |
| | Prozin |
| Fluphenazine | Prolixin |
| Fluphenazine decanoate (monthly | Prolixin decanoate |
| injection) | Siqualone decanoate |
| | Modecate |
| | Moditen |
| Haloperidol | Haldol |
| | Serenase |
| Haloperidol decanoate (monthly | Haldol decanoate |
| injection) | |
| Lurasidone | Latuda |
| Olanzapine | Zyprexa |
| | Arkolamyl |
| | Olazax |
| | Rolyprexa |
| | Zalasta |
| | Zapilux |
| Olanzapine pamoate (every 2 weeks | Zyprexa |
| injection) | Zypadhera |
| Olanzapine pamoate (monthly | Zyprexa Relprevv |
| injection) | Zypadhera |
| Paliperidone | Invega |
| Paliperidone palmitate (monthly | Invega Sustenna |
| injection) | |
| Paliperidone palmitate (every 3 | Invega Trinzia |
| months injection) | |
| Perphenazine | Trilafon |
| Quetiapine (including XR | Seroquel |
| formulation) | |
| Risperidone LA (every 2 weeks | Risperdal Consta |
| injection) | |
| Trifluoperazine | Stelazine |
| Ziprasidone^{a} | Geodon |

^{a}These medications may increase the QT interval and may be allowed if approved by the Sponsor or designee.
Baseline QTcF must be ≤450 msec for males or ≤470 msec for females.

### Study Population

A planned total of approximately 400 subjects 13 years of age or older with a diagnosis of schizophrenia who have had an inadequate response to antipsychotic medications will be enrolled to yield a minimum of 300 randomized subjects into the Efficacy Analysis Set, at a 1:1 allocation ratio (valbenazine versus placebo).

Human subjects will be screened for potential enrollment and, if qualified, enrolled in the study. Inclusion criteria and exclusion criteria for the study are set forth below. Subjects must fulfill all inclusion and exclusion criteria to participate in the study.

### Inclusion Criteria

Subjects must meet all the following inclusion criteria:
1. Completed written informed consent for adult subjects or written and witnessed pediatric assent from the subject and written informed consent from the subject's legal guardian in accordance with the Institutional Review Board (IRB)/Independent Ethics Committee (IEC) and according to local laws and regulations.
2. At the time of signing the informed consent (or assent for pediatric subjects), subject must be ≥13 years of age with a body weight of at least 50 kg.
3. Medically confirmed diagnosis of schizophrenia as defined by the MINI Version 7.0.2 for Psychotic Disorders for the DSM-5 for subjects ≥18 years of age, inclusive; or K-SADS-PL for subjects 13 to 17 years of age.
4. The initial diagnosis of schizophrenia must be ≥1 year prior to screening.
5. The subject is receiving background antipsychotic therapy (other than clozapine) at a total daily dose between 4 mg and 8 mg of risperidone equivalents (outlined in Table 2), as confirmed by pharmacy records and, if available, additional records/documentation (e.g., medical/prescription records and/or a letter from a treating physician). Concomitant treatment with a subtherapeutic dose of a second antipsychotic is permissible if used to treat specific symptoms, such as insomnia or anxiety, but not if it is used for refractory positive psychosis symptoms.
   The total combined antipsychotic dose must remain between 4 mg and 8 mg daily dose of risperidone equivalent.
   In subjects aged 13 to 17 years, a lower risperidone equivalent dose may be acceptable following consultation with Sponsor or designee for approval prior to enrollment.
6. Plasma levels for at least 1 of the subject's antipsychotic medications must be detectable by an available assay (Table 3).
7. The subject is treated with a stable regimen antipsychotic medication for treatment of schizophrenia with no clinically meaningful change (no increase in dose, ≤25% decrease in dose for tolerability) in the prescribed dose for ≥3 weeks before screening (as confirmed by pharmacy records (and medical records if available), between screening and Day 1, and no anticipated dose adjustment throughout study participation up to Week 10.
   If the subject is taking a long-acting injectable antipsychotic, no dose change within 8 weeks prior to Screening, during the Screening period, or expected throughout study participation.
8. Must meet all the following criteria at screening and Day 1 (Visit 2):
   Positive and Negative Symptom Scale (PANSS) total score ≥70
   PANSS score of ≥4 on at least 1 of the following:
      P1 (delusions)
      P3 (hallucinations)
      P6 (suspiciousness)
      G9 (unusual thought content)
   Clinical Global Impression of Severity (CGI-S) score ≥4
   Stable background antipsychotic medication dose (≤25% change in risperidone equivalent total daily dose) between screening and Day 1
   Stable PANSS total score (≤15% change) between screening and Day 1
9. The subject is outpatient with stable symptomatology ≥3 weeks prior to screening (e.g., no hospitalizations for schizophrenia, no increase in level of psychiatric care due to worsening of symptoms of schizophrenia).
10. The subject's diagnosis, background antipsychotic therapy, and severity of symptoms must be confirmed by the Sponsor or designee prior to the first dose of study treatment on Day 1.
11. The subject must have an adult informant (e.g., a family member, social worker, caseworker, residential facility staff, or nurse) who meets the following requirements:
   Spends ≥2 hours/week with the subject
   Is able to provide input for the clinicians completing study rating scales, including the PANSS
   Is able and willing to provide written informed consent. Informant consent is separate from the subject's written informed consent (or assent for minors) and must be signed before the subject will be considered eligible for the study
   Is able to participate in at least 1 in-person or video interview at the study site during the screening period and able to provide continuing input by attending corresponding onsite visits or participating in telephone interviews
   Is considered reliable by the investigator
12. A body mass index (BMI) of 18.0 to 40.5 kg/m2 (inclusive) at screening (BMI is defined as the subject's weight in kg divided by the square of the subject's height in meters).
13. Female subjects of childbearing potential must have a negative serum β-human chorionic gonadotropin (β hCG) pregnancy test at screening and a negative urine pregnancy test at Day 1.
14. Female subjects of childbearing potential must agree to use contraception consistently from screening until 30 days after the last dose of study drug or final study visit, whichever is longer. A female who is not of childbearing potential must meet 1 of the following:
   Postmenopausal, defined as no menses for 12 months without an alternative medical cause and confirmed by elevated follicle-stimulating hormone (FSH) consistent with a postmenopausal range
   Permanent sterilization procedure, such as hysterectomy, bilateral salpingectomy, or bilateral oophorectomy
Acceptable methods of contraception include the following:
   Intrauterine device (IUD) or intrauterine hormone-releasing system (IUS)
   Combined (estrogen and progestogen containing) hormonal contraception associated with inhibition of ovulation, which may be oral, intravaginal, or transdermal at least 3 months prior to screening
   Progestogen-only hormonal contraception associated with inhibition of ovulation, which may be oral, injected, or implanted at least 3 months prior to screening.
   Bilateral tubal ligation
   Total abstinence from sexual intercourse (periodic abstinence is not acceptable)
   Sexual partner(s) who had been vasectomized at least 3 months prior to screening with medically confirmed successful procedure
15. Male subjects must agree to use contraception consistently from screening until 30 days after last dose of study treatment. The acceptable method of contraception for male subjects are:
   Condom with spermicide (cream, spray, foam, gel, suppository, or polymer film)
   Vasectomy at least 3 months prior to screening with medically confirmed successful procedure
   Total abstinence from sexual intercourse (periodic abstinence is not acceptable)
16. Willing to comply with all study procedures and restrictions; and in the opinion of the investigator, the subject is capable of understanding and complying with all study procedures and restrictions. This criterion must be reconfirmed prior to the first dose of study treatment on Day 1.

### Exclusion Criteria

Subjects will be excluded from the study if they meet any of the following criteria:
1. Pregnant or breastfeeding or plans to become pregnant during the study. This criterion must be reconfirmed prior to the first dose of study treatment on Day 1.
2. Known hypersensitivity to any component of the formulation of valbenazine.
3. Have comorbid Parkinsonism (drug-induced or otherwise) as assessed by the investigator or exhibit more than a minimal level of extrapyramidal signs/symptoms, as documented by a score on the modified Simpson Angus Scale (SAS; excluding #10, akathisia) >6 at screening.
4. Has history of treatment resistant schizophrenia, as defined by any of the following criteria:
   History of clozapine treatment for treatment resistant psychosis; or
   History of multiple adequate and failed antipsychotic medication trials where subject demonstrated minimal or no improvement (failure to tolerate a medication does not constitute failure to respond)
5. Diagnosis of schizoaffective disorder; a lifetime diagnosis of bipolar disorder; or a lifetime diagnosis of obsessive-compulsive disorder based on the MINI Version 7.0.2. Subjects with a historical prior lifetime diagnosis of schizoaffective disorder may be enrolled in the study with Sponsor or designee approval provided that the investigator can attest that the subject's overall history and current clinical presentation and history is most consistent with schizophrenia, not schizoaffective disorder.
6. Recent (within the last 6 months prior to screening) occurrence of panic disorder, major depressive episode, or other comorbid psychiatric conditions currently requiring clinical attention based on the MINI Version 7.0.2. This criterion must be reconfirmed prior to the first dose of study treatment on Day 1.
7. Evidence of depression as measured by a Calgary Depression Scale for Schizophrenia (CDSS) score >8 at screening and Day 1.
8. Initiation or changes in nonpharmacological psychosocial therapeutic treatment (e.g., day hospital treatment, cognitive-behavioral therapy) within 3 weeks before screening or expected to change throughout the length of the study.
9. Considered by the investigator to be at imminent risk of suicide or injury to self, others, or property, or the subject has attempted suicide within 1 year prior to screening (Visit 1). Subjects who have positive answers on item numbers 4 or 5 on the Columbia-Suicide Severity Rating Scale (C-SSRS; since last visit) at Day 1 are excluded.
10. Diagnosis of moderate or severe substance use disorder (with the exception of nicotine or caffeine dependence) within the 6 months prior to screening based on the MINI.
11. Positive alcohol test or urine drug screen for disallowed substances, including amphetamines; barbiturates; cocaine; marijuana; methadone; methamphetamine; 3,4-methylenedioxymethamphetamine (MDMA); phencyclidine; or nonprescribed benzodiazepines or opiates.
   Note: Subjects testing positive for marijuana at screening may be eligible for participation in the study provided that the investigator's clinical assessment indicates that the subject is not a regular user of marijuana, and, after counseling, the subject agrees to not use marijuana for the duration of the study. Under this circumstance, a local urine dipstick drug screen must be performed at the Day 1 visit and verified to be negative prior to conducting any other study procedures at that visit
12. Have a clinically significant unstable medical condition within 60 days prior to screening in the judgment of the investigator (30 days prior to screening for minor medical conditions) or any laboratory value outside the normal range that is considered by the investigator to be clinically significant at the screening visit.
13. Have any known history of long QT syndrome or cardiac arrhythmia.
14. Have a triplicate average electrocardiogram (ECG) QT interval corrected for heart rate using Fridericia's formula (QTcF) of >450 msec (male subjects) or >470 msec (female subjects) or the presence of any clinically significant cardiac abnormality during the screening period.
15. Have a history of severe hepatic impairment or chronic elevation of any of the following laboratory tests:
   Serum creatinine >1.5×the upper limit of normal (ULN)
   Aspartate aminotransferase (AST)≥2.5×ULN
   Alanine aminotransferase (ALT)≥2.5×ULN
   Gamma-glutamyl transferase (GGT)≥3.0×ULN
   Total bilirubin>1.5 mg/dL. Subjects with a documented diagnosis of Gilbert's syndrome are not required to meet the bilirubin criteria
16. Any of the following laboratory abnormalities at screening:
   Hemoglobin<10 g/dL
   White blood cell (WBC) count<3.0×10³/mm³
   Platelet count<100,000/mm³
   Absolute neutrophil count<1.0×10³/mm³
17. Have a hematologic malignancy or solid tumor diagnosed within 3 years prior to Screening or not in remission, with the exception of localized skin cancer or carcinoma in situ of the cervix that has been excised.
18. Have any known history of neuroleptic malignant syndrome.
19. Are currently taking any of the prohibited medications. Subjects who have received these medications in the past, must have been off them for at least 30 days prior to the screening visit.
20. Has previously participated in this study; has used any active investigational drug in the context of a clinical trial within the preceding 30 days or <5 half-lives prior to screening, whichever is longer; has participated in more than 2 clinical studies within 12 months prior to screening; or is currently participating in another clinical trial per subject database query; or has participated in a clinical study for a psychiatric condition that is exclusionary per this protocol. This criterion must be reconfirmed prior to the first dose of study treatment on Day 1.
21. Prior (within 6 months of Screening) or concomitant use of any VMAT2 inhibitor (i.e., valbenazine, reserpine, tetrabenazine, deutetrabanzine); or a history of intolerance to VMAT2 inhibitors.
22. Any reason that makes the subject unsuitable for participation in this study (e.g., subject is homeless, known to have difficulty complying with treatment or medical procedures, known to provide inaccurate medical information, or attempt participation in clinical trials inappropriately).

### Investigational Product, Dosage, and Mode of Administration

Valbenazine (valbenazine tosylate, NBI-98854) is a selective, orally active vesicular monoamine transporter 2 (VMAT2) inhibitor developed by Neurocrine Biosciences, Inc. (NBI). Valbenazine was approved by the US Food and Drug Administration (FDA) in April 2017 for the treatment of adults with tardive dyskinesia (TD), under the trade name INGREZZA®.

Valbenazine appears to cause little or no cytochrome P450 (CYP) enzyme inhibition or induction at pharmacologically relevant concentrations. Valbenazine is a moderate inhibitor of P-glycoprotein, but only at concentrations that could be achieved in the gastrointestinal tract and is not an inhibitor of a panel of other drug transporters. Valbenazine is metabolized by hydrolysis to NBI-98782 ([+]-α-dihydrotetrabenazine). NBI-98782 is subsequently metabolized in part by CYP2D6. Valbenazine and NBI-98782 both have the ability to bind to and inhibit VMAT2. However, NBI-98782 is the most potent, and is believed to be responsible for the majority of the observed pharmacological activity of VMAT2 inhibition. Repeat-dose nonclinical toxicology studies conducted in mice, rats, and dogs revealed no adverse effects at doses of 60 mg/kg/day in the mouse, 3 mg/kg/day in the rat and 15 mg/kg/day in the dog. The no observed adverse effect level (NOAEL) in the central nervous system, respiratory and cardiovascular safety pharmacology studies resulted in systemic exposures to valbenazine and NBI-98782 that were above those achieved at the highest proposed therapeutic dose in humans. Valbenazine had a modest negative effect on rat fertility at 10 mg/kg/day (NOAEL of 3 mg/kg/day). The NOAEL for embryo/fetal development in rats and rabbits was 15 mg/kg/day and 50 mg/kg/day, respectively. There was no evidence of teratogenicity in rats or rabbits. Valbenazine was negative in genotoxicity assays (in vitro Ames and chromosomal aberration assays and an in vivo rat micronucleus test). Valbenazine administration did not increase tumors in carcinogenicity studies in rats or hemizygous Tg.rasH2 mice.

As of the data cutoff for the Investigator's Brochure (10 Apr. 2021), 33 clinical studies of valbenazine conducted by NBI have been completed with final data available: 16 Phase 1 studies in healthy subjects or special populations; 7 Phase 2 or 3 studies in subjects with TD, 2 Phase 4 studies in subjects with TD, and 8 Phase 1b, 2, or 2b studies in subjects with Tourette Syndrome (TS) (1 in adults; 6 in children and adolescents; and 1 in children, adolescents, and adults). Two Phase 3 studies in subjects with Huntington Disease (HD) are ongoing.

Valbenazine 20, 40, 60, and 80 mg oral capsules have been used in the clinical development programs, and valbenazine has been administered at single doses up to 300 mg and multiple doses up to 100 mg.

The most common adverse reactions identified in the valbenazine clinical development program of adults with TD (in 3 placebo-controlled studies of 6-week treatment duration reported in ≥2% of subjects and at a higher percentage in valbenazine vs placebo) include somnolence (10.9%; somnolence, fatigue, sedation), anticholinergic effects (5.4%; dry mouth, constipation, disturbance in attention, vision blurred, urinary retention), and balance disorders/fall (4.1%; fall, gait disturbance, dizziness, balance disorder). Common treatment-emergent adverse events (TEAEs) in pediatric subjects with TS with incidence >5% in the valbenazine treatment groups and with a higher incidence than placebo include headache, somnolence, sedation, upper respiratory tract infection, insomnia, fatigue, vomiting, and suicidal ideation. In the entire valbenazine clinical development program as of 10 Apr. 2021, treatment-emergent serious adverse events (SAEs) occurring in 3 or more valbenazine-treated subjects each were schizophrenia, suicidal ideation, chronic obstructive pulmonary disease, syncope, abdominal pain, depression, mental status changes, and schizoaffective disorder.

Ten deaths have been reported in completed and ongoing valbenazine studies conducted by NBI: 9 occurred in adult subjects with TD and 1 adult subject with HD. Eight deaths occurred in subjects taking valbenazine, 1 occurred in a subject taking placebo, and 1 remains blinded. All of the deaths were considered not related or unlikely related to study drug.

Valbenazine may prolong the QT interval, although the degree of QT prolongation is not clinically significant at concentrations expected with recommended dosing. In subjects taking a strong CYP2D6 or CYP3A4 inhibitor, or who are CYP2D6 poor metabolizers, valbenazine concentrations may be higher and QT prolongation clinically significant.

No cardiovascular, laboratory, or vital sign related safety signals have been identified. Increases in serum prolactin above normal laboratory ranges have been noted; mean changes in subjects who received placebo were considerably smaller. Valbenazine does not appear to increase suicidality.

Valbenazine will be supplied in 20 mg and 40 mg capsules. Valbenazine can be taken with or without food. The valbenazine doses in this study are 40 mg and 80 mg and are based on the commercially approved doses in adults with tardive dyskinesia (TD), clinical studies in adults with TD, ongoing studies in Huntington Disease (HD) and pediatric studies (6 to 18 years old) in patients with Tourette Syndrome (TS). In Phase 3 studies in adults with TD, the starting dose was 40 mg, with titration up to 80 mg. The proposed doses of valbenazine (40 mg and 80 mg QD) were selected based on cumulative valbenazine pharmacokinetic(s) (PK), safety, tolerability, and efficacy data.

The dose optimization scheme, based on tolerability and safety assessments, will allow each subject to receive an optimized dose during the Stable Dose Period.

### Reference Therapy, Dose, and Mode of Administration

Matching placebo capsules identical in appearance to the investigational drug valbenazine will be orally administered in a double-blind manner (2 placebo capsules) on an identical schedule as those receiving valbenazine. Placebo can be taken with or without food.

### Study Treatment

Study treatments are summarized in Table 4.

TABLE 4
Study Treatments

| Treatment Group | Valbenazine | Placebo |
|---|---|---|
| Treatment administration | Valbenazine will be orally | Placebo is administered in the |
| | self-administered by subjects | same manner and on an identical |
| | once per day | schedule as valbenazine |
| Unit dose strength | 20 mg or 40 mg capsules | Matching placebo capsules |
| Dose level | 40, and 80 mg | NA |
| Dose formulation | Capsule | Matching placebo capsules |
| Route of administration | Oral | Oral |
| Sourcing | Provided centrally by Sponsor | Provided centrally by Sponsor |
| Packaging and labeling | Blister packs containing | Matching placebo will be |
| | 17 doses (2 capsules per dose). | provided in the same manner as |
| | Study drug labelling will be in | the active treatment. |
| | accordance with applicable | |
| | regulatory requirements. | |

### Study Treatment Administration

Study treatment will be self-administered at home starting on Day 1 (Visit 2); study treatment adherence will be monitored with a mobile technology system. Study treatment should be administered at approximately the same time in the afternoon or evening each day during the study. If a subject forgets or is unable to take the study treatment on a given day, the subject should not take 2 doses within the same day. The subject should resume normal dosing the following day.

Valbenazine will be supplied in 20 mg and 40 mg capsules. Subjects may take valbenazine with or without food.

Matching placebo capsules identical in appearance to the investigational drug valbenazine will be orally administered in a double-blind manner (2 placebo capsules) on an identical schedule as those receiving valbenazine.

All subjects will receive placebo for 2-weeks during the Placebo Run-in Period. Subjects will receive randomized study treatment QD starting at the end of Week 2 (Visit 3) until the end of the Randomized Double-Blind Treatment Period (end of Week 10; Visit 6). Subjects will receive study treatment for a total of 10 weeks (2 weeks of placebo and 8 weeks of randomized study treatment).

### Study Treatment and Antipsychotic Therapy Adherence

Because nonadherence with medication is a significant challenge in schizophrenia trials, this study will use a medication adherence mobile application for all subjects in the study to monitor adherence to study treatment and oral antipsychotic medication. The platform is provided on a smartphone application and uses artificial intelligence to confirm ingestion of study treatment. Built-in reminders and a communication system allow real-time intervention in case of missed doses. Use of the platform is required for all subjects in the study to reinforce the proper dosing schedule and improve data integrity.

### (i) Study Treatment Adherence

Adherence to study treatment (valbenazine or placebo) will be monitored with the medication adherence mobile application throughout the blinded Placebo Run-In Period (Day 1 through end of Week 2) and the Randomized Double-Blind Treatment Period (Week 3 through the end of Week 10).

Nonadherence with study treatment includes subjects who are <75% adherent between visits (including nonadherence as assessed via the digital adherence technology). Subjects who are consistently nonadherent with study treatment medication should be discussed with the Medical Monitor or may be discontinued from study treatment at the Sponsor's discretion.

### (ii) Antipsychotic Therapy Adherence

During screening, adherence to subject's background antipsychotic medications will be evaluated based on review of data from a patient or caregiver report, blood sample collections, and clinical or pharmacy records.

### (a) Medication Adherence Application: Antipsychotic Medication

The medication adherence mobile application will also be used to monitor adherence to background daily oral antipsychotic medications throughout this study (Screening [Visit 1] to end of Week 10 [Visit 6]). Subjects should be instructed to answer the questionnaire about background medication use every day.

Subjects using a daily oral antipsychotic must be registered in the platform at screening (Visit 1) and begin confirming appropriate adherence to their background antipsychotic to ensure adherence prior to Day 1. Subjects using a long-acting injectable antipsychotic must be registered in the platform at screening (Visit 1) and will use it only to record study treatment intake. At Day 1 (Visit 2), all subjects will begin using the platform to monitor study treatment intake and continue to log their background antipsychotic use.

Subjects using a long-acting injectable antipsychotic will use the medication adherence mobile application to record study treatment intake only.

### (b) Pharmacy Records

Recent adherence to background antipsychotic medication(s) (i.e., in the 2 to 6 consecutive months before screening) will be assessed based on review of available pharmacy records. During the screening period, sites must obtain the past 2 months of available pharmacy records; in addition, sites will be asked to obtain >2 to ≤6 consecutive months of available pharmacy records (optional).

### (c) Blood Samples for Antipsychotic Medications

A blood sample to assess whether antipsychotic medications (see Table 3) are at detectable levels will be obtained at screening and the time point(s) indicated in Table 5.

TABLE 5
Schedule of Assessments

| | Procedure | | | | | | |
|---|---|---|---|---|---|---|---|
| | Screening | | | | | | |
| | Period | Placebo | | Randomized Double- | | | Follow-up/ET |
| | (2 to | Run-In | | Blind Treatment Period | | | (14 days after |
| | 4 weeks) | (2 weeks) | | (8 weeks) | | | last dose)^{a} |
| | Week | | | | | | |
| | −4 | | End of | End of | End of | End of | End of |
| | through | | W2 | W4 | W6 | W10 | W12 |
| | Day −1 | Day 1 | (±3 days) | (±3 days) | (±3 days) | (±3 days) | (±3 days) |
| | Visit | | | | | | |
| | 1 | 2 | 3 | 4 | 5 | 6 | 7 |
| Informed consent^{b} | X | | | | | | |
| Informant informed consent^{c} | X | | | | | | |
| Subject database check^{d} | X | | | | | | |
| Mobile technology | X | X | | | | | |
| system training | | | | | | | |
| Inclusion/exclusion criteria | X | X | | | | | |
| Placebo-control reminder script^{e} | X | X | X | X | X | X | |
| Medical history | X | | | | | | |
| AE monitoring | X | X | X | X | X | X | X |
| Prior and | X | X | X | X | X | X | X |
| concomitant medications | | | | | | | |
| Randomization | | | X | | | | |
| Dispense study treatment | | X | X | X | X | | |
| Study treatment dosing^{f} | | X | X | X | X | | |
| Study treatment accountability^{g} | | | X | X | X | X | |
| Physical examination^{h} | X | | | | | X | |
| Vital signs | X | X | X | X | X | X | X |
| 12-lead ECGⁱ | X | X | | X | | X | |
| Pregnancy test^{j} | X (s) | X (u) | X (u) | X (u) | X (u) | X (u) | X (u) |
| Serum prolactin | | X | | X | | X | |
| Clinical laboratory tests^{k} | X | X | | X | | X | |
| Urinalysis | X | X | | X | | X | |
| Urine drug screen | X | X | | | | | |
| and alcohol test ^{l} | | | | | | | |
| Blood for antipsychotic | X | | | | | X | |
| analysis | | | | | | | |
| Blood for PK assessments^{m} | | X | | X | | X | |
| Blood for genotyping | | X | | | | | |
| C-SSRS | X | X | X | X | X | X | X |
| CDSS | X | X | | | | X | |
| AIMS | | X | | | | | |
| MINIⁿ | X | | | | | | |
| K-SADS-PL^{o} | X | | | | | | |
| Simpson Angus Scale | X | X | | | | X | |
| PANSS | X | X | X | X | X | X | |
| PSP scale | | X | X | | | X | |
| Healthcare Utilization | | X | | | X | X | |
| Resource Questionnaire | | | | | | | |
| CGI-S | X | X | X | X | X | X | |
| EQ-5D-5L/EQ-5D-Y^{p} | | X | X | | | X | |
| SDS | | X | X | | | X | |

AE = adverse event;
AIMS = Abnormal Involuntary Movement Scale;
CDSS = Calgary Depression Scale for Schizophrenia;
CGI = clinical global impression;
CGI-S = clinical global impression of severity;
C-SSRS; Columbia-Suicide Severity Rating Scale;
ECG = electrocardiogram;
EQ-5D-5L = EuroQol 5 Dimensions 5 Levels;
EQ-5D-Y = EuroQol 5 Dimensions Youth;
ET = early termination;
K-SADS-PL = Kiddie Schedule for Affective Disorders and Schizophrenia, Present and Lifetime version;
MINI = Mini International Neuropsychiatric Interview;
PANSS = Positive and Negative Syndrome Scale;
PK = pharmacokinetic;
PSP = personal and social performance;
s = serum;
SDS = Sheehan Disability Scale;
u = urine
^{a}Final study visit for subjects who complete the study (or early termination).
^{b}Informed consent (or assent for minors, per local requirements) must be obtained before any study procedures can be performed. Subject database informed consent will also be obtained.
^{c}Must be obtained at screening (Visit 1) or during the Screening Period. The initial discussion with the informant may include an oral informed consent.
^{d}Should be performed as soon as all necessary identifiers have been collected.
^{e}Administered to both the subject and informant.
^{f}Study subjects will be instructed to take study treatment QD in the evening with or without food.
^{g}Subjects will return all unused study treatment at each study visit.
^{h}Physical examination will include measurement of weight without shoes and height (at screening only).
ⁱA standard 12-lead ECG will be conducted in triplicate (at least 1 minute apart and within 15 minutes) after the subject has rested supine for at least 5 minutes. The ECG parameters that will be assessed include heart rate, QT, QTcF, PR intervals, and QRS duration based on the ECG machine readings (QTcF may need to be calculated).
^{j}Pregnancy tests are required for all females of childbearing potential. A serum pregnancy test will be conducted at screening. A urine pregnancy test will be conducted at all subsequent onsite visits.
^{k}Clinical laboratory tests include hematology, clinical chemistry, and urinalysis. All blood samples will be obtained under non-fasted conditions.
^{l} May be repeated at any time at the investigator's clinical judgment. Any positive urine drug screens or alcohol btests during conduct of the study must be discussed with the Sponsor or designee to determine the subject's disposition.
^{m}PK sample on Day 1 must be predose.
ⁿFor subjects 18 years of age or older.
^{o}For subjects under 13 to 17 years of age.
^{p}The EQ-5D-5L will be administered to subjects ≥18 years old and the EQ-5D-Y will be administered to subjects 13 to 17 years old.

### Subject Restrictions

### Prior and Concomitant Medications

All prescription and over-the-counter medications, dietary supplements (including vitamins), and herbal supplements taken by the subject within 30 days before screening (Visit 1) will be recorded on the electronic case report form (eCRF). Other psychotropic medications taken within the last year will be recorded on the eCRF. Any additions or changes in the dose of these medications will be entered on the eCRF with indication, dose, route, and dates of drug administration.

Medications to treat psychiatric and medical conditions: All coexistent diseases or conditions should be treated in accordance with prevailing medical practice. All medications should be on a stable treatment regimen (including no changes to the dose and frequency of ongoing medications and no discontinuation of medications) for a minimum of 21 days before screening procedures. Benzodiazepines must be at a stable dose for 2 weeks before screening.

Prohibited medications: The following medication classes are prohibited from 30 days prior to screening (Visit 1) (unless otherwise stated) until the final study visit (or early termination) as noted in Table 6.

TABLE 6
Classes of Prohibited Concomitant Medications

| Medication Classes | Example Medications |
|---|---|
| CYP3A4 Strong Inducers | Phenytoin, phenobarbital, rifabutin, rifampin, |
| | primidone, St. John's Wort, Carbamazepine |
| Dopamine agonists and | Ropinirole, pramipexole, carbidopa/levodopa |
| precursors | |
| MAOIs | Isocarboxazid, phenelzine, selegiline, |
| | tranylcypromine |
| Stimulants | amphetamine, methylphenidate, and ephedrine |
| VMAT2 Inhibitors | Deutetrabenazine, tetrabenazine, and reserpine |

CYP3A4 = cytochrome P450 3A4;
MAOI = monoamine oxidase inhibitors;
VMAT2 = vesicular monoamine transporter 2

Time-restricted medications: The medication classes summarized in Table 7 are restricted from screening through the end of Week 10. Any episodic usage of time-restricted medications should be documented, including the date and time of last dose taken prior to study visits. The documentation of time-restricted medications applies to as needed treatment for anxiety or agitation, extrapyramidal motor symptoms, or insomnia. The subject's prescribed daily treatment regimen is exempt from this requirement; however allowable dosage ranges must be followed. As specified in Table 7, episodic use of time-restricted medications should be ≥4 hours before the start of any primary or key secondary efficacy assessment at Visits 2 through 6, or early termination (ET) visit.

Benzodiazepines must be at a stable dose equal to or less than the equivalent of 3 mg/day of lorazepam for 2 weeks before screening. As needed use of benzodiazepines is restricted; benzodiazepines should not be administered 4 hours prior to the subject's clinic visit (Table 7).

Additional detailed guidance on excluded/allowed medications may be provided in separate reference documentation provided to study sites.

TABLE 7
Time-Restricted Classes of Concomitant Medications: Allowable
Chronic or Episodic Use from Screening to End of Week 10

| Medication Class^{a} | Dosage Allowed |
|---|---|
| Anticholinergics | Maximum of 3 mg/day benztropine or equivalent for chronic |
| (including antihistamine use for | use |
| anticholinergic properties) | Maximum of 2 mg/day benztropine or equivalent for episodic |
| | use; up to 2 times per week |
| Benzodiazepines | Maximum of 3 mg/day of lorazepam or another |
| | benzodiazepine for chronic use |
| | Maximum of 2 mg/day of lorazepam or another |
| | benzodiazepine for episodic use; up to 3 days per week |
| Non-Benzodiazepine Hypnotics | Zolpidem up to 10 mg/day, zaleplon up to 20 mg/day, |
| | eszopiclone up to 3 mg/day, or zopiclone 7.5 mg/day or |
| | doxepin up to 6 mg/day for sleep |
| Opioid Analgesics | Only episodic use is allowed with Sponsor or designee |
| | approval |
| Medications known to increase QT | Allowed with Sponsor or designee approval |
| interval^{b} | |

Note:
Any usage of time-restricted medications should be documented, including the date of last dose prior to study Visits 2 through 6.
^{a}A listing of time-restricted medications is provided in Table 8.
^{b}The use of medication known to increase the QT interval may be allowed if approved by the Sponsor or designee. Baseline QTcF must be ≤450 msec for males or ≤470 msec for females.

The names of time-restricted concomitant medications in this study include, but are not limited to, those medications listed in Table 8. The dosing limit of some of these medications during the Randomized Double-Blind Treatment Period are provided in Table 7. The timing restriction of episodic use (as needed) of some of these medications prior to efficacy assessments during the Randomized Double-Blind Treatment are described in herein.

TABLE 8
List of Time-Restricted Concomitant Medications

| Medication Class | Name of Medications |
|---|---|
| Anticholinergics (including | Benztropine |
| antihistamines with Anti- | Biperidin |
| cholinergic properties) | Cyclobenzaprine |
| | Diphenhydramine |
| | Hydroxyzine |
| | Procyclidine |
| | Trihexyphenidyl |
| Benzodiazepines | Alprazolam |
| | Clonazepam |
| | Clorazepate |
| | Flurazepam |
| | Lorazepam |
| | Oxazepam |
| | Temazepam |
| | Triazolam |
| Medications that may prolong the QT | Antipsychotics |
| interval. | Chlorpromazine |
| | Thioridazine |
| The use of medication known to increase | Pimozide |
| the QT interval may be allowed if | Ziprasidone |
| approved by the Sponsor or designee. | Antidepressants |
| Baseline QTcF must be ≤450 msec for | Citalopram |
| males or ≤470 msec for females. | Antibiotics |
| | Azithromycin |
| | Clarithromycin |
| | Erythromycin |
| | Moxifloxacin |
| | Antiarrhythmics |
| | Quinidine |
| | Procainamide |
| | Amiodarone |
| | Sotalol |
| Non-Benzodiazepine Hypnotics | Eszopiclone |
| | Zaleplon |
| | Zolpidem |
| | Zopiclone |
| Opioid Analgesics | Alfentanil |
| | Fentanyl (including |
| | transdermal) |
| | Hydrocodone |
| | Hydromorphone |
| | Meperidine |
| | Morphine |
| | Oxycodone |
| | Oxymorphone |
| | Pentazocine |
| | Propoxyphene |
| | Sufentanil |
| | Tramadol |

### Discontinuation of Study Treatment

If a subject prematurely discontinues study treatment, the investigator will record the reason for discontinuation on the relevant source document and eCRF. Data for any outcome measures, particularly the primary and secondary endpoints, as well as safety follow-up, are important to collect. Subjects that discontinue treatment will continue to participate in the study and attend all remaining visits. Subjects should not be withdrawn from the study due to treatment discontinuation, regardless of study treatment adherence, unless consent/assent for study participation has been withdrawn.

Reasons for discontinuation from study treatment include but are not limited to:
Withdrawal from study treatment by subject/caregiver
Death
Lost to follow-up
Site termination by the Sponsor
Study termination by the Sponsor
AE
Pregnancy
Protocol deviation
Sponsor decision

The investigator must discontinue study treatment if the subject experiences any of the following:
If the type, frequency, or severity of any AE becomes unacceptable/intolerable
If the subject is unable to tolerate the lowest allowable dose
QTcF of >500 msec (cardiologist verified) on any ECG tracing
If the subject is confirmed to be pregnant
Withdrawal of consent/assent for study treatment administration by parent/caregiver or subject

### End of Study Definition

Primary Completion: The primary completion date is defined as the date when the last subject is assessed or receives an intervention for the final collection of data used for the primary endpoint(s), whether the study concluded as planned or was terminated early. The planned primary completion date for this study is the date when the last subject has completed the assessments for Week 10 (Visit 6). If the study is terminated early, then the primary completion date will be the date of the last visit for the last subject in the study.

End of Study: The end of study is defined as the date of the last visit of the last subject or last scheduled procedure shown in the Schedule of Assessments for the last subject in the study globally.

### Endpoints

Primary Efficacy Endpoint: Change in PANSS total score from Baseline to Week 10.

Key Secondary Efficacy Endpoint: Change in CGI-S score from Baseline to Week 10.

Additional Secondary Efficacy Endpoint: Change in Personal and Social Performance Scale (PSP) score from Baseline to Week 10.

Other Endpoints: (1) Change in VAS scores for the EuroQol 5 Dimensions 5 Levels (EQ-5D-5L; subjects ≥18 years old) or EQ-5D Youth (EQ-5D-Y; subjects 13 to 17 years old) from baseline to Week 10. (2) Change in Sheehan Disability Score (SDS) from baseline to Week 10.

### Pharmacokinetics

Blood samples for analysis of plasma valbenazine and metabolite concentrations will be collected during the study.

### Safety Assessments

Safety and tolerability will be monitored throughout the study. Safety will be assessed through the occurrence of adverse events (AEs), clinical laboratory tests (hematology, serum chemistry, and urinalysis), vital sign measurements (including orthostatic blood pressure and pulse rate), physical examinations (including weight), 12-lead electrocardiogram (ECG), Columbia-Suicide Severity Rating Scale (C-SSRS), Calgary Depression Scale for Schizophrenia (CDSS), and Simpson Angus Scale (SAS).

Concomitant medications and AEs will be monitored throughout the study as described herein.

For any abnormal safety assessment deemed clinically significant, the investigator will perform appropriate follow-up assessments (e.g., repeat analysis), until the cause of the abnormality is determined and/or until the value returns to baseline (or within normal limits) or the investigator deems the abnormality to be of no clinical significance.

Appropriate psychiatric evaluation and intervention will be provided for any study treatment-emergent suicidal behavior or clinically significant suicidal ideation as determined by the investigator.

### Vital Sign Measurements

Vital signs will be measured for the following: orthostatic systolic and diastolic blood pressure, orthostatic pulse rate, respiratory rate, and body temperature. Blood pressure will be measured using a calibrated automatic blood pressure cuff after the subject has been supine for at least 5 minutes and after approximately 2 minutes of standing.

At screening, vital sign measurements will be obtained before any scheduled blood sample collection.

### Medical History

A medical and psychiatric history will be taken at the screening visit and as needed throughout the study.

Physical Examination (including height and weight)

The complete physical examination will consist of an assessment of general appearance, skin and mucosae, head, eyes, ears, nose, throat, neck (including thyroid), lymph nodes, chest/lungs, cardiovascular, abdomen, extremities, musculoskeletal, and neurological system. Weight will also be measured. Weight and height (measured only at screening) will be measured with subjects not wearing shoes, and weight will be measured with subjects not wearing outerwear (e.g., jackets or coats).

### Electrocardiogram

A standard 12-lead ECG will be conducted in triplicate (at least 1 minute apart and within 15 minutes) after the subject has rested supine for at least 5 minutes. The ECG parameters that will be assessed include heart rate, PR interval, QRS duration, QT interval, and QT interval corrected for heart rate using Fridericia's correction (QTcF) (machine readings or calculated). Additionally, the occurrence of de- and re-polarization and rhythm disorders or other abnormalities will be assessed. Based on the review of these parameters, the investigator will note if the ECG is Normal, Abnormal not Clinically Significant, or Abnormal Clinically Significant. If the ECG is Abnormal Clinically Significant, the investigator will provide a description of the abnormality recorded on the AE eCRF.

### Clinical Laboratory Assessments

All clinical laboratory assessments will be performed by a central laboratory. The central laboratory will provide instructions and supplies to the study staff before study initiation and instructions will be included in a laboratory manual. The following clinical safety laboratory assays will be performed:

Hematology: complete blood count including white blood count (WBC) with differential, red blood cell (RBC) count, hemoglobin, hematocrit, and platelet count, mean corpuscular hemoglobin (MCH), mean corpuscular hemoglobin concentration (MCHC), mean corpuscular volume (MCV), red cell distribution width (RDW), mean platelet volume (MPV).

Clinical Chemistry: sodium, potassium, calcium, magnesium, phosphorus, chloride, blood urea nitrogen, bicarbonate, creatinine, uric acid, albumin, alkaline phosphatase, lactate dehydrogenase, AST, ALT, gamma-glutamyl transferase, creatine kinase, total bilirubin, total protein, total cholesterol, triglycerides, and non-fasting glucose.

Urinalysis: casts, crystals, specific gravity, nitrite, ketones, protein, urobilinogen, glucose, bilirubin, leukocyte esterase, occult blood, and pH; microscopic examination of sediment will be performed only if the results of the urinalysis dipstick evaluation are positive for nitrite, protein, leukocyte esterase, or blood.

The following additional laboratory tests will be performed:

Urine Drug Screen and Alcohol Test: The urine drug screen (UDS) will test for amphetamines, barbiturates, cocaine, marijuana, methadone, methamphetamine, 3,4-methylnedioxymethamphetamine (MDMA), phencyclidine, and nonprescribed benzodiazepines or opiates. The UDS will be performed at screening by the central laboratory.

An alcohol test will be performed at the study site at screening and Day 1.

Prolactin: Serum prolactin samples will be shipped to a central laboratory for analysis. Prolactin results will remain blinded to investigators, subjects, CROs, and Sponsor until database lock. The DMC may review unblinded prolactin data during the course of the study.

Pregnancy Test: Pregnancy tests will be performed throughout the study for female subjects of childbearing potential. A serum beta-human chorionic gonadotropin (β-hCG) pregnancy test will be performed at screening and a urine pregnancy test (using a urine pregnancy kit provided by the central laboratory) will be performed at the timepoints indicated in Table 5.

### CYP2D6 Status

CYP2D6 phenotyping will be conducted to understand its influence on PK, safety, and efficacy of valbenazine. A blood sample will be collected from enrolled subjects for the analysis of CYP2D6 status (i.e., normal, intermediate, poor, or ultrarapid metabolizers) on Day 1. Approximately 4 mL of blood will be collected in tubes containing dipotassium ethylenediaminetetraacetic acid (EDTA K₂). After the sample is obtained, it should be thoroughly mixed. The vials will be stoppered and labeled with the study barcode and subject number. The collection and submission of medical information will be accomplished with strict adherence to professional standards of confidentiality. Genotyping blood samples collected from subjects will be shipped to a central laboratory for analysis.

### Pharmacokinetic Assessments

### Blood Samples for Antipsychotic Medications

A blood sample to the assess whether antipsychotic medications are at detectable levels will be obtained at screening and the time point(s) indicated in Table 5. As part of inclusion into the study, subjects must have detectable levels of background antipsychotic medication at screening. Once a subject is randomized, an additional sample will be taken at the end of Week 10 (Visit 6) to assess adherence to a subject's background antipsychotic medication. Maximum blood volume collected for this assessment will be no more than 4 mL.

### Blood Samples for Valbenazine PK

Blood samples for determination of plasma concentrations of valbenazine and metabolites will be collected at the timepoints identified in the Schedule of Assessments (see Table 5).

For each sample, approximately 2 mL of blood will be collected in tubes containing dipotassium ethylenediaminetetraacetic acid (EDTA K₂). The exact time of sampling in hours and minutes will be recorded for all pharmacokinetic (PK) blood samples. A PK sample should be collected from subjects who withdraw from the study early. The blood samples will be processed and stored according to the procedure as specified in the laboratory manual. Samples will be shipped on dry ice to the central laboratory for storage. The central laboratory will ship the samples to a PK laboratory for analysis.

### Adverse Events

All AEs, whether observed by the investigator, reported by the subject, noted from laboratory findings, or identified by other means, will be recorded from the time the subject has signed the ICF until the subject's final study visit.

An AE is any untoward medical occurrence in a patient or clinical investigation subject administered a pharmaceutical product which does not necessarily have a causal relationship with the study treatment. An AE can therefore be any unfavorable and unintended sign (including an abnormal laboratory finding, for example), symptom, or disease temporally associated with the use of a medicinal product, whether considered related to the medicinal product.

AEs include, but are not limited to, any of the following:
Worsening or change in nature, severity, or frequency of conditions present at the start of the study
Subject deterioration beyond what would be expected due to the primary illness
Intercurrent illness
Drug interaction

If at any time after Visit 2 the subject's response to the suicidal ideation section of the C-SSRS is worse than the Visit 2 assessment, it will be documented as an AE. All suicidal behaviors will be documented as an AE.

Subjects or their parent/caregiver should be questioned in a general way, without asking about the occurrence of any specific symptom. The investigator should attempt to establish a diagnosis of the event based on signs, symptoms, and/or other clinical information. In such cases, the diagnosis should be documented as the AE and not the individual signs/symptoms. Following questioning and evaluation, all AEs, whether believed by the investigator to be related or unrelated to the study treatment, must be documented in the subject's medical records, in accordance with the investigator's normal clinical practice and on the AE eCRF. Each AE is to be evaluated for duration, intensity, frequency, seriousness, outcome, other actions taken, and relationship to the study treatment.

The following are not considered AEs:
Continuous persistent disease/symptom present before study treatment administration, unless it unexpectedly progresses, or increases in severity following study treatment administration
Study treatment failure or lack of efficacy
Pregnancy
Overdose of either study treatment or concomitant medication without any clinical signs or symptoms

### Intensity of Adverse Events

Adverse events must be graded for intensity. An intensity category of mild, moderate, or severe, as defined in Table. It should be noted that the term “severe” used to grade intensity is not synonymous with the term “serious.”

TABLE 9
Intensity of Adverse Events

| Grade | Intensity |
|---|---|
| Mild | An AE that is usually transient and may require only minimal treatment or |
| | therapeutic intervention. The event does not generally interfere with usual activities |
| | of daily living. |
| Moderate | An AE that is usually alleviated with additional specific therapeutic intervention. |
| | The event interferes with usual activities of daily living, causing discomfort but |
| | poses no significant or permanent risk of harm to the research subject. |
| Severe | An AE that interrupts usual activities of daily living, or significantly affects clinical |
| | status, or may require intensive therapeutic intervention. |

AE = adverse event.

### Relationship to Study Treatment

The investigator will document their opinion of the relationship of the AE to treatment with study treatment using the criteria outlined in Table 10.

TABLE 10
Relationship of Adverse Events to Study Treatment

| Relationship | Description |
|---|---|
| Definite | The AE follows a reasonable temporal sequence from administration of the study treatment, |
| | abates upon discontinuation of the study treatment, follows a known or hypothesized cause- |
| | effect relationship, and (if appropriate) reappears when the study treatment is reintroduced. |
| Possible | The AE follows a reasonable temporal sequence from administration of the study treatment |
| | and cannot be reasonably explained by the known characteristics of the subject's clinical |
| | state, environmental, or toxic factors, or other modes of therapy administered to the subject. |
| | There should be some evidence to support a causal relationship between the study treatment |
| | and the AE. |
| Unlikely | The temporal sequence between the AE and the study treatment is such that the study |
| | treatment is not likely to have any reasonable association with the AE or other plausible |
| | explanations exist for the AE (e.g., disease, other drugs). |
| Not related | The AE does not follow a reasonable temporal sequence from administration of the study |
| | treatment, may not abate upon discontinuation of the study treatment, does not follow a |
| | known or hypothesized cause-effect relationship, and (if applicable) may not reappear when |
| | the treatment is reintroduced, furthermore, there may exist a clear alternative medical |
| | explanation (e.g., underlying disease state) or association with study procedure or study |
| | conduct. |

AE = adverse event.

### Serious Adverse Events

All serious adverse events (SAEs) will be recorded from the time the subject has signed the ICF until the final study visit. If the investigator learns of any SAE, including a death, at any time after a subject has been withdrawn from or has completed the study, and the investigator considers the event to be reasonably related to the study intervention or study participation, the investigator must promptly notify the Sponsor.

### Definition of a Serious Adverse Event

An SAE is any AE that results in any of the following outcome:
Death
A life-threatening AE. Life-threatening means that the subject was, in the view of the investigator or Sponsor, at immediate risk of death from the reaction as it occurred. It does not mean that hypothetically the event might have caused death if it occurred in a more serious form
Inpatient hospitalization or prolongation of existing hospitalization. Hospitalization for elective treatment or a preexisting condition that did not worsen during the clinical investigation is not considered an AE. Hospitalization or nursing home admission for the purpose of caregiver respite is not considered an AE. Complications that occur during hospitalization are AEs, and if a complication prolongs hospitalization, the event is considered serious. Treatment in a hospital emergency room is not a hospitalization
A persistent or significant incapacity or substantial disruption of a person's ability to conduct normal life functions
A congenital anomaly/birth defect
Important medical events that may not result in death, be life-threatening, or require hospitalization. These events may be considered serious when, based on appropriate medical judgment, they may jeopardize the health of the subject and may require medical or surgical intervention to prevent one of the outcomes listed. Any other event thought by the investigator to be serious should also be reported, following the reporting requirements detailed in this section. Examples of such medical events include allergic bronchospasm requiring intensive treatment in an emergency room or at home, blood dyscrasias, convulsions that do not result in inpatient hospitalization, or the development of drug dependency or drug abuse

### Statistical Methods

Provided herein is a brief description of the planned primary analysis of the primary and secondary objectives.

### Statistical Hypothesis

The null hypothesis for the primary endpoint is that there is no difference between the valbenazine group and the placebo group in the change in PANSS total score from analysis baseline to Week 10 in the Efficacy Analysis Set. In other words, H₀: μ₁=μ₂, where μ₁ is the change from analysis baseline in PANSS total score in the placebo group at Week 10 and μ₂ is the change from analysis baseline in PANSS total score in the valbenazine treatment group at Week 10. The alternative hypothesis is that the valbenazine group is different than the placebo group in the change from analysis baseline in PANSS total score at Week 10 (i.e., H₁: μ₁≠μ₂).

### Statistical Estimands

The primary estimand uses a hypothetical strategy to estimate the effect of the initially randomized treatment (valbenazine versus placebo) described as if all subjects had adhered to treatment and remained in the study. Consistent with the International Council for Harmonisation of Technical Requirements for Pharmaceuticals for Human Use (ICH) E9 Addendum, the 4 attributes are:
Population:
   Subjects with a diagnosis of schizophrenia as defined by the study inclusion/exclusion criteria and who demonstrate study treatment compliance (i.e., ≥75% compliance between Visit 2 and Visit 3), no study treatment dose reduction during the Placebo Run-In Period, and symptom stability (i.e., ≤15% change in PANSS scores between Visits 1 and 3) during the blinded Placebo Run-In Period.
Endpoints:
   Primary: Change in PANSS total score from Baseline to Week 10.
   Key Secondary: Change in CGI-S score from Baseline to Week 10.
Intercurrent events are considered to be a part of the treatments being compared.
Summary measures:
   Primary: Difference of means in change from baseline in PANSS total score at Week 10 between valbenazine and placebo groups.
   Key Secondary: Difference of means in change from baseline in CGI-S score at Week 10 between valbenazine and placebo groups.

All available assessments from Visits 4, 5, and 6 will be used, regardless of occurrence of intercurrent events.

### Sample Size Determination

The planned sample size of approximately 300 subjects (in the Efficacy Analysis Set) randomized approximately 1:1 to valbenazine versus placebo is based on a power calculation for the primary endpoint. Based on a 2-sample t-test, a sample size of 150 subjects per treatment group will have approximately 80% power to detect a treatment difference corresponding to an effect size of 0.375 at a 2-sided 5% significance level, assuming a 20% drop-out rate.

The effect size is based on analysis results for change in PANSS total score in subjects with schizophrenia found in the literature. The standard deviation for PANSS total score ranges ˜8 to 12 and a standard deviation of 10 is assumed for these calculations. A treatment difference of ˜3 to 4 in change from baseline in PANSS total score is considered clinically meaningful for adjunctive treatment.

Only those subjects who have stable PANSS total score (≤15% change between Visit 1 and Visit 3) and are compliant to study treatment (≥75% compliance) during the placebo run-in period, have had no study treatment dose reduction during the Placebo Run-In Period, and maintain stable background antipsychotic dose (<25% change) during the Screening and Placebo Run-In Periods, will be included in the Efficacy Analysis Set. Since it is anticipated that ˜25% of enrolled subjects will not qualify for the Efficacy Analysis Set, the study is planned to enroll ˜400 subjects to yield the 300 subjects needed for the primary analysis.

An unblinded interim analysis will take place when 180 subjects (in the Efficacy Analysis Set) have had the opportunity to complete 8 weeks of randomized treatment (Week 10). At this time, the sample size will be re-estimated based on the promising zone methodology. If the results at the interim analysis look promising, the sample size may be increased to maintain conditional power of 80% at the final analysis. The overall total sample size for analysis may be increased up to a maximum of 440 subjects in the Efficacy Analysis Set (approximately 587 enrolled). The foregoing sample size and power estimates were obtained from nQuery® Advisor Version 8.

### Analysis Sets

Statistical analysis sets used in this study are defined in Table 11. Additional analysis sets may be specified in the Statistical analysis plan (SAP).

TABLE 11
Analysis Sets

| Population | Description |
|---|---|
| Efficacy Analysis Set | The Efficacy Analysis Set includes all randomized subjects with |
| | stable PANSS scores (i.e., PANSS total score at Visit 3 is ≤15% |
| | different than at Visit 1), study treatment adherence (i.e., ≥75% |
| | compliance between Visit 2 and Visit 3), no study treatment |
| | dose reduction during the Placebo Run-In, and who maintained |
| | their background antipsychotic dose (<25% change between |
| | Visit 1 and Visit 3), with baseline and relevant postbaseline |
| | data. Subjects will be analyzed according to their randomized |
| | treatment group, regardless of adherence to study treatment |
| | administration. |
| Safety Analysis Set | The safety analysis set will include all randomized subjects who |
| | take at least 1 dose of study treatment and have any postbaseline |
| | safety data. Subjects will be analyzed according to their |
| | randomized treatment group, unless they receive the incorrect |
| | study treatment for the entire treatment duration. |

### Statistical Analyses

Descriptive statistical methods will be used to summarize the data from this study. Descriptive statistics typically include the number of subjects (n), mean, standard deviation (SD) or standard error (SE), median, first (Q1) and third (Q3) quartile, minimum, maximum, and confidence intervals for continuous variables; and refers to the number and percentage of subjects for categorical variables. Descriptive statistics will be presented by treatment group for the PANSS total score observed values and changes from baseline at each visit during the double-blind, randomized treatment period.

Descriptive statistics for the key secondary endpoint of change from baseline in CGI-S score will be presented by treatment group at each postbaseline visit. Treatment comparisons for the change from baseline to the end of Week 10 will be analyzed similarly to the primary endpoint. The key secondary endpoint will only be tested for statistical significance if the primary endpoint is considered statistically significant at the 0.05 significance level.

The analysis set that will be used for the primary efficacy analysis will include all subjects in the Efficacy Analysis Set which will include all randomized subjects who met study treatment compliance criteria between Visits 2 and 3; demonstrated symptom stability between Visits 1 and 3; have limited dose change in antipsychotic medication (<25% change in dose between Visits 1 and 3); have no study treatment dose reduction during the Placebo Run-In Period; and had at least 1 efficacy assessment. All available data will be included in the primary analyses, including data collected after early discontinuation of study treatment.

Safety assessments will be summarized descriptively by study treatment. All subjects randomized and who receive at least 1 dose of study treatment will be included in the safety analysis.

The planned sample size of approximately 300 subjects (in the Efficacy Analysis Set) randomized approximately 1:1 to valbenazine vs placebo is based on a power calculation for the primary endpoint. Based on a 2-sample t-test, a sample size of 150 subjects per treatment group will have approximately 80% power to detect a treatment difference corresponding to an effect size of 0.375 at a 2-sided 5% significance level, assuming a 20% drop-out rate.

### Efficacy Analyses

All efficacy analyses will use the data collected after the Placebo Run-in Period and immediately before the first dose of randomized treatment as the baseline value.

The summary of planned statistical methods for the primary analysis of the primary and secondary endpoints is as follows.

### (i) Procedure to Control for Multiple Comparisons

Sequential testing will be performed between primary and key secondary endpoints.

The key secondary endpoint will be tested only if the primary endpoint is considered statistically significant at the 5% significance level.

All other p-values will not be adjusted for multiplicity and should be considered nominal p-values.

### (ii) Primary Endpoint

The primary endpoint of the change from baseline in PANSS total score at Week 10 will be analyzed using a linear mixed-effects repeated measures model using the scores at the end of Weeks 4, 6, and 10. The model will include the baseline PANSS total score as a covariate and region (US versus non-US), age (<18 years, 18 to 40 years, and >40 years), treatment group (valbenazine or placebo), visit, and treatment group-by-visit interaction as fixed effects. Subject will be included as a random effect. Significance tests will be based on least-squares means using a two-sided significance level of 0.05. The primary comparison will be the contrast between treatment groups at Week 10.

The Efficacy Analysis Set will be used in the primary analysis.

(iii) Key Secondary Endpoints

Treatment comparisons for the change from baseline to the end of Week 10 in CGI-S score will be analyzed similarly to the primary endpoint using the available postbaseline values (Weeks 4, 6, and 10).

The Efficacy Analysis Set will be used for the primary analysis.

### (iv) Additional Secondary Endpoints

The continuous secondary endpoint measuring change from analysis baseline in PSP score at Week 10 will be analyzed similarly to the primary endpoint using the available postbaseline values (Weeks 4, 6, and 10).

The Efficacy Analysis Set will be used for the primary analysis for all secondary endpoints.

### Safety Analyses

Safety data from this study will be analyzed using the safety analysis set. The subject incidence of treatment-emergent AEs will be tabulated by treatment group for AEs, SAEs, fatal AEs, and AEs leading to discontinuation of study treatment. Descriptive statistics by treatment group will be generated for additional safety data, including selected laboratory analytes, vital signs, ECG parameters, C-SSRS, and SAS, etc.

### Interim Analyses

There is one planned interim analysis in the study, as well as ongoing monitoring of the study, as described in herein.

An unblinded interim analysis will be conducted prior to the completion of randomization and when the results of the first 60% (N=180) randomized subjects (in the Efficacy Analysis Set) have had the opportunity to complete 8 weeks (i.e., Week 10) of randomized treatment. At this time, the sample size will be re-estimated based on the promising zone methodology. If the results at the interim analysis look promising, the sample size may be increased to maintain conditional power of 80% at the final analysis. The interim analysis will include a nonbinding (unblinded to the independent data monitoring committee [DMC] only) futility analysis using the conditional power approach. In addition, an unblinded sample size re-estimation will be performed based on the promising zone approach (Mehta and Pocock (2011)). Based on the interim analysis results, the overall total sample size for analysis may increase up to a maximum of 440 randomized subjects in the Efficacy Analysis Set (approximately 587 enrolled). In the event of a sample size increase, a step function will be utilized to maintain study integrity. At the final analysis, the primary endpoint will be tested using the Cui-Hung-Wang (CHW) weighted combination statistic. See, e.g., Cui L, Hung H M, and Wang S J. Modification of sample size in group sequential clinical trials. Biometrics. 1999 September; 55(3):853-7.

### Example 2: Formulations/Study Medications

The composition of 20, 40, 60, and 80 mg dose strength valbenazine is provided in the tables below.

TABLE 12
Formulation 1: Formulation 1: Quantitative Composition of Valbenazine
Capsules, 20, 40, 60, and 80 mg (Free Base Equivalent)

| Component | % (w/w) |
|---|---|
| Valbenazine ditosylate | 40.0 |
| Silicified microcrystalline cellulose | 25.0 |
| Isomalt | 20.0 |
| Hypromellose | 5.0 |
| Partially pregelatinized maize starch | 7.5 |
| Magnesium stearate | 2.5 |
| Total | 100 |
| Hard gelatin capsule | 1 unit |

TABLE 13
Formulation 2: Quantitative Composition of Valbenazine
Capsules, 40 mg (Free Base Equivalent)

| Component | % (w/w) |
|---|---|
| Valbenazine ditosylate | 28.2 |
| Mannitol | 61.8 |
| Partially pregelatinized maize starch | 7.7 |
| Fumed silica | 1.2 |
| Magnesium stearate | 1.0 |
| Total | 100 |
| Hard gelatin capsule | 1 unit |

Although the disclosure has been described with reference to the above examples, it will be understood that modifications and variations are encompassed within the spirit and scope of the disclosure. The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method for the add-on treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, wherein the subject is also being administered at least one co-therapeutic agent for the treatment of schizophrenia.

2. The method of claim 1, wherein the subject has at least one sign or symptom of schizophrenia.

3. The method of claim 1 or 2, wherein the subject has at least one positive, negative, and/or cognitive sign or symptom of schizophrenia.

4. The method of claim 2 or 3, wherein the at least one sign or symptom is delusions, hallucinations, disorganized speech, disorganized behavior or attention, anhedonia, catatonic behavior, affective flattening, alogia, avolition, conceptual disorganization, or any combination thereof.

5. A method for the add-on treatment of schizophrenia, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, wherein the subject has at least one symptom chosen from the following symptoms in Positive and Negative Symptom Scale (PANSS): P1 (delusions), P3 (hallucinations), P6 (suspiciousness), and G9 (unusual thought content), and further wherein the subject is also being administered at least one co-therapeutic agent for the treatment of schizophrenia.

6. The method of claim 5, wherein the subject has a total PANSS score ≥70.

7. The method of claim 5 or 6, wherein the subject has Clinical Global Impression of Severity (CGI-S) score ≥4.

8. The method of any one of claims 5-7, wherein the subject has a stable background antipsychotic medication dose.

9. The method of any one of claims 5-8, wherein the subject has a stable PANSS total score.

10. The method of any one of claims 1-9, wherein the subject is residually symptomatic or has at least one residual sign or symptom after first- or second-line treatment of schizophrenia.

11. The method of any one of claims 1-10, wherein the subject is residually symptomatic or has at least one residual sign or symptom after mono- or combination therapy for schizophrenia.

12. The method of claim 11, wherein the mono- or combination therapy is initial therapy.

13. The method of any one of claims 1-12, wherein the at least one co-therapeutic agent is least one antipsychotic agent.

14. The method of claim 13, wherein the antipsychotic agent is a typical antipsychotic agent.

15. The method of claim 14, wherein the typical antipsychotic agent is benperidol, chlorpromazine, droperidol, flupentixol, fluphenazine, haloperidol, levomepromazine, loxapine, molindone, pericyazine, perphenazine, pimozide, prochlorperazine, promazine, sulpiride, thiothixene, trifluoperazine, thioridazine, zuclopenthixol, or any combination thereof.

16. The method of claim 13, wherein the antipsychotic agent is an atypical antipsychotic agent.

17. The method of claim 16, wherein the atypical antipsychotic agent is clozapine, olanzapine, risperidone, sertindole, quetiapine, paliperidone, asenapine, ziprasidone, surmontil, iloperidone, aripiprazole, or any combination thereof.

18. The method of any one of claims 13-17, wherein the subject is receiving concomitant treatment with a subtherapeutic dose of a second antipsychotic agent.

19. The method of claim 18, wherein the concomitant treatment with a subtherapeutic dose of a second antipsychotic is used to treat insomnia or anxiety.

20. The method of claim 19, wherein the concomitant treatment with a subtherapeutic dose of a second antipsychotic is used to treat symptoms other than refractory positive psychosis symptoms.

21. The method of any one of claims 13-20, wherein the subject has an inadequate response to the at least one antipsychotic agent.

22. The method of any one of claims 13-21, wherein the subject is on at least a second clinical use of the at least one antipsychotic agent.

23. The method of any one of claims 1-22, wherein the subject is also being administered risperidone and/or a risperidone equivalent.

24. The method of claim 23, wherein the subject is also being administered a total daily dose of from about 4 mg to about 8 mg of risperidone and/or risperidone equivalents.

25. The method of any one of claims 1-24, wherein the subject is receiving background antipsychotic therapy at a total daily dose of from about 4 mg to about 8 mg of risperidone equivalents according to Table 2.

26. The method of claim 25, wherein the subject has a stable antipsychotic agent dose.

27. The method of claim 26, wherein the stable antipsychotic agent dose is defined as ≤25% change in risperidone equivalent total daily dose.

28. The method of any one of claims 13-27, wherein the subject is also being administered up to two antipsychotic agents.

29. The method of claim 28, wherein the total combined antipsychotic dose is from about 4 mg to about 8 mg daily dose of risperidone equivalents according to Table 2.

30. The method of any one of claims 13-29, wherein the antipsychotic agent is other than clozapine.

31. The method of any one of claims 13-30, wherein the subject is treated with a stable regimen of antipsychotic agent(s) with no clinically meaningful change in the prescribed dose.

32. The method of claim 31, wherein the no clinically meaningful change in the prescribed dose is no increase in dose or ≤25% decrease in dose for tolerability.

33. The method of claim 32, wherein no dose adjustment is anticipated.

34. The method of any one of claims 13-33, wherein the subject is also being administered a long-acting injectable antipsychotic.

35. The method of claim 34, wherein there is no dose change in the long-acting injectable antipsychotic.

36. The method of any one of claims 21-35, wherein the inadequate response comprises at least one baseline criterion selected from:
Positive and Negative Symptom Scale (PANSS) total score of ≥70;
PANSS score of ≥4 on at least one symptom selected from P1 (delusions), P3 (hallucinations), P6 (suspiciousness), and G9 (unusual thought content);
Clinical Global Impression of Severity (CGI-S) score of ≥4;
stable background antipsychotic medication dose of ≤25% change in risperidone equivalent total daily dose according to Table 2; and
a stable PANSS total score of ≤15% change.

37. The method of any one of claims 1-36, wherein the subject has a confirmed diagnosis of schizophrenia as defined by the MINI Version 7.0.2 for Psychotic Disorders for the Psychotic Disorders for the Diagnostic and Statistical Manual of Mental Disorders, Fifth Edition (DSM-V) for a subject ≥18 years of age or K-SADS-PL for a subject 13 to 17 years of age.

38. The method of any one of claims 1-37, wherein the subject has a confirmed initial diagnosis of schizophrenia for at least one year.

39. The method of any one of claims 1-38, wherein the subject is at least 13 years of age with a body weight of at least 50 kg.

40. The method of any one of claims 1-39, wherein the subject is an outpatient with stable symptomatology.

41. The method of any one of claims 1-40, wherein the subject does not have comorbid Parkinsonism and/or does not or exhibit more than a minimal level of extrapyramidal signs or symptoms as defined by a score on the modified Simpson Angus Scale (SAS; excluding #10, akathisia) >6.

42. The method of any one of claims 1-41, wherein the subject does not have treatment-resistant schizophrenia, as defined by at least one of the following criteria:
a history of clozapine treatment for treatment-resistant psychosis; and/or
a history of multiple adequate and failed antipsychotic medication trials, wherein the subject demonstrated minimal or no improvement.

43. The method of any one of claims 1-42, wherein the subject does not have a diagnosis of schizoaffective disorder; a lifetime diagnosis of bipolar disorder; a lifetime diagnosis of obsessive-compulsive disorder; a recent occurrence of panic disorder, a depressive episode, and/or other comorbid psychiatric condition(s) requiring clinical attention based on the MINI Version 7.0.2.

44. The method of any one of claims 1-43, wherein the subject does not have evidence of depression as measured by a Calgary Depression Scale for Schizophrenia (CDSS) score >8.

45. The method of any one of claims 1-44, wherein the subject has not attempted suicide and/or does not have positive answers on item numbers 4 or 5 on the Columbia-Suicide Severity Rating Scale (C-SSRS).

46. The method of any one of claims 1-45, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered via a titration scheme that comprises the up-titration of the (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester over a period of no more than about six weeks until an optimized dose is administered.

47. The method of claim 46, wherein the titration scheme comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof at an initial dose equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily for about two weeks, provided that the patient tolerates the initial dose and that the patient has not had an adequate response, increasing the dose and administering the increased dose to the subject.

48. The method of claim 47, wherein the increased dose is equivalent to about 60 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

49. The method of claim 47, wherein the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

50. The method of any one of claims 46-49, wherein the titration scheme further comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof at the increased dose for about two weeks.

51. The method of any one of claims 46-49, wherein if the subject does not tolerate the increased dose, the optimized dose is the initial dose.

52. The method of claim 50, wherein if the subject tolerates the increased dose and if the subject has had an adequate response, the optimized dose is the increased dose.

53. The method of claim 51 or 52, further comprising administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof to the subject.

54. The method of claim 50, wherein if the subject tolerates the increased dose and if the subject has not had an adequate response, the method further comprises increasing the dose.

55. The method of claim 54, wherein the further increased dose is equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

56. The method of claim 54 or 55, wherein if the subject does not tolerate the further increased dose, the optimized dose is the increased dose.

57. The method of claim 54 or 55, wherein if the subject tolerates the further increased dose and if the subject has had an adequate response, the optimized dose is the further increased dose.

58. The method of claim 56 or 57, further comprising administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof to the subject.

59. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered in the afternoon or evening.

60. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject is treated with a stable regimen of antipsychotic agent(s) with ≤25% change in risperidone equivalent total daily dose.

61. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject is treated with a stable regimen of antipsychotic agent(s) with no clinically meaningful change in the prescribed dose.

62. The method of claim 51, wherein the no clinically meaningful change in the prescribed dose is no increase in dose or a ≤25% decrease in dose for tolerability in the prescribed dose for ≥3 weeks.

63. The method of claim 52, wherein no dose adjustment is anticipated.

64. The method of any one of the preceding claims, wherein the subject is also being administered a long-acting injectable antipsychotic with no dose change within 8 weeks prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof.

65. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a Diagnostic and Statistical Manual of Mental Disorders, Fifth Edition (DSM-V) diagnosis of schizophrenia.

66. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a confirmed diagnosis of schizophrenia as defined by the MINI Version 7.0.2 for Psychotic Disorders for the Diagnostic and Statistical Manual of Mental Disorders, Fifth Edition (DSM-V) for a subject ≥18 years of age or K-SADS-PL for a subject 13 to 17 years of age.

67. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a confirmed initial diagnosis of schizophrenia for at least one year.

68. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has at least one symptom chosen from the following symptoms in Positive and Negative Symptom Scale (PANSS): P1 (delusions), P3 (hallucinations), P6 (suspiciousness), and G9 (unusual thought content).

69. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a total PANSS score ≥70.

70. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a stable PANSS total score with ≤15% change.

71. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a Clinical Global Impression of Severity (CGI-S) score ≥4.

72. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has a stable background antipsychotic medication dose with ≤25% change in risperidone equivalent total daily dose according to Table 2.

73. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject is an outpatient with stable symptomatology for ≥3 weeks.

74. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject does not have treatment-resistant schizophrenia, as defined by at least one of the following criteria:
a history of clozapine treatment for treatment-resistant psychosis; and/or
a history of multiple adequate and failed antipsychotic medication trials, wherein the subject demonstrated minimal or no improvement.

75. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject does not have a diagnosis of schizoaffective disorder; a lifetime diagnosis of bipolar disorder; a lifetime diagnosis of obsessive-compulsive disorder; a recent occurrence of panic disorder, a depressive episode, and/or other comorbid psychiatric condition requiring clinical attention based on the MINI Version 7.0.2.

76. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject does not have evidence of depression as measured by a Calgary Depression Scale for Schizophrenia (CDSS) score >8.

77. The method of any one of the preceding claims, wherein prior to administration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof, the subject has not attempted suicide within one year and/or does not have positive answers on item numbers 4 or 5 on the Columbia-Suicide Severity Rating Scale (C-SSRS).

78. The method of any one of the preceding claims, wherein the add-on treatment results in a change in total PANSS score from Analysis Baseline to Week 10; a change in CGI-S of illness from Analysis Baseline to Week 10, a change in Personal and Social Performance (PSP) score from Analysis Baseline to Week 10, a change in VAS scores for the EuroQol 5 Dimensions 5 Levels (EQ-5D-5L; subjects ≥18 years old) or EQ-5D Youth (EQ-5D-Y; subjects 13 to 17 years old) from baseline to Week 10; and/or a change in Sheehan Disability Score (SDS) from baseline to Week 10.

79. The method of any one of the preceding claims, wherein the treatment results in a reduction in the subject's CGI-S positive symptoms, relative to the subject's score at baseline.

80. The method of any one of the preceding claims, wherein the treatment results in a change in EuroQol 5 Dimensions 5 Levels (EQ-5D-5L) scores from Analysis Baseline to Week 10 and/or a change in Sheehan Disability Score (SDS) from Analysis Baseline to Week 10.

81. The method of any one of claims 1-80, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in a solid dosage form.

82. The method of any one of claims 1-81, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is orally administered.

83. The method of any one of claims 1-82, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in the form of a capsule.

84. The method of any one of claims 1-83, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered daily.

85. The method of any one of claims 1-84, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily or twice daily.

86. The method of any one of claims 1-85, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily.

87. The method of any one of claims 1-86, wherein the therapeutically effective amount is an amount equivalent to from about 10 mg to about 90 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

88. The method of any one of claims 1-87, wherein the therapeutically effective amount is an amount equivalent to from about 20 mg to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

89. The method of any one of claims 1-88, wherein the therapeutically effective amount is an amount equivalent to about 20 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

90. The method of any one of claims 1-89, wherein the therapeutically effective amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

91. The method of any one of claims 1-89, wherein the therapeutically effective amount is an amount equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

92. The method of any one of claims 1-89, wherein the therapeutically effective amount is an amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

93. The method of any one of claims 1-89, wherein the therapeutically effective amount is an amount equivalent to about 40 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

94. The method of any one of claims 1-89, wherein the therapeutically effective amount is an amount equivalent to about 60 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

95. The method of any one of claims 1-89, wherein the therapeutically effective amount is an amount equivalent to about 80 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

96. The method of any one of claims 1-95, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a free base.

97. The method of any one of claims 1-95, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a salt.

98. The method of any one of claims 1-97, wherein the salt is a tosylate salt.

99. The method of claim 98, wherein the tosylate salt is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt of structural Formula (I):

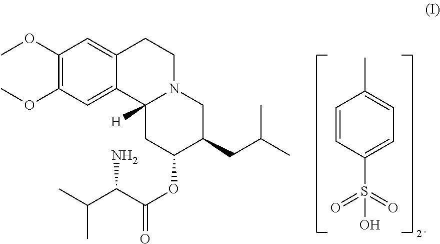

100. The method of any one of claims 1-99, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is in crystalline form.

101. The method of claim 100, wherein the crystalline form of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is Form I of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt having a differential scanning calorimetric (DSC) peak temperature within 2% of 243° C.

102. The crystalline form of claim 100 or 101, wherein the DSC peak temperature is within 1% of 243° C.

103. The crystalline form of any one of claims 100-102, wherein the DSC peak temperature is within 0.5% of 243° C.

104. The crystalline form of any one of claims 100-103, wherein the crystalline form has an X-ray powder diffraction (XRPD) pattern comprising a peak at a two-theta angle of 6.3°±0.2°.

105. The crystalline form of any one of claims 100-104, wherein the crystalline form has an X-ray powder diffraction (XRPD) pattern comprising a peak at a two-theta angle of 17.9°±0.2°.

106. The crystalline form of any one of claims 100-105, wherein the crystalline form has an X-ray powder diffraction (XRPD) pattern comprising a peak at a two-theta angle of 19.7°±0.2°.

107. The crystalline form of any one of claims 100-106, wherein the crystalline form is stable upon exposure to about 25° C. and about 60% relative humidity.

108. The crystalline form of any one of claims 100-107, wherein the crystalline form has a D90 particle size of about 70 μM in length.

109. The crystalline form of any one of claims 100-107, wherein the crystalline form has a D10 particle size of about 10 μM in length.

110. The crystalline form of any one of claims 100-109, wherein the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt.

111. The crystalline form of any one of claims 100-110, wherein the crystalline form has a purity of no less than 98% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt.

112. The crystalline form of any one of claims 100-111, wherein the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester tosylate salt; and has an X-ray powder diffraction (XRPD) pattern comprising peaks at two-theta angles of 6.3°±0.2°, 17.9°±0.2°, and 19.7°±0.2°.
